(19) United States
(12) Patent Application Publication
(10) Pub. No.: US 2006/0063210 A1
(43) Pub. Date: Mar. 23, 2006

(54) Histone deacetylase whole cell enzyme assay

(75) Inventors: Zuomei Li, Kirkland (CA); Jeffrey M. Besterman, Baie D'Urfe (CA); Claire Bonfils, Montreal (CA)

Correspondence Address: JOSEPH C. ZUCCHERO, SUITE 1200, 500 WEST CUMMINGS PARK, WOBURN, MA, 01801, US

(73) Assignee: Methylgene, Inc.

(21) Appl. No.: 11/231,528

(22) Filed: Sep. 21, 2005

Related U.S. Application Data
(60) Provisional application No. 60/611,964, filed on Sep. 22, 2004.

(51) Int. Cl. C12Q 1/34; G01N 33/542; G01N 33/53

(52) U.S. Cl. 435/7.9; 435/18

(57) ABSTRACT

The invention relates to enzymatic assays for protein deacetylases. More particularly, the invention relates to such assays utilizing whole cells. The invention provides assays which allow assessment of the level of a protein deacetylase activity in whole cells taken directly from the body of the mammal.

### RELATED APPLICATIONS

This application claims the benefit of U.S. Provisional Application Ser. No. 60/611,964, filed on Sep. 22, 2004, which is incorporated herein, in its entirety, by reference.

### BACKGROUND OF THE INVENTION

1. Field of the Invention

The invention relates to enzymatic assays for protein deacetylases. More particularly, the invention relates to such assays utilizing primary intact whole cells.

2. Summary of the Related Art

Histone deacetylases play an important role in gene regulation in mammalian cells. Gray and Ekstrom, Expr. Cell. Res. 262: 75-83 (2001); Zhou et al., Proc. Natl. Acad. Sci. USA 98: 10572-10577 (2001); Kao et al. J. Biol. Chem. 277: 187-193 (2002) and Gao et al. J. Biol. Chem. 277: 25748-25755 (2002) teach that there are 11 members of the histone deacetylase (HDAC) family. Another family of deacetylases involved in gene expression is the Sir2 family. Gray and Ekstrom, supra, teach that there are seven members of the Sir2 family in humans.

The role of HDACs in transcription and its link to diseases, such as cancer has recently been explored. Minnucci et al., Proc. Natl. Acad. Sci. USA 94: 11295-11300 (1997); Hassig et al., Chem. Biol. 4: 783-789 (1998); Grignani et al., Nature 391: 815-818 (1998) and Siddique et al., Oncogene 16: 2283-2285 (1998) suggest that inhibitors of HDACs may be useful for transcription therapy in various human diseases.

As efforts at developing HDAC inhibitors for therapeutic treatment progresses, there is a need for assays to determine the activity of such inhibitors. Lechner et al., Biochim. Biophys. Acta 1296: 181-188 (1996) teaches the use of tritylated, acetylated histones as a substrate. Taunton et al., Science 272: 408-411 (1996) teaches the use of tritylated, acetylated synthetic peptides derived from histones as substrate. These assays proved difficult to standardize.

More recently, non-isotopic assays have been developed. Heltweg and Jung, Journal of Biomolecular Screening 8: 89-95 (2003) describes an assay using the fluorescent compound MAL (Boc-LysAc-AMC) and a partially purified rat liver HDAC in the presence or absence of the HDAC inhibitor trichostatin A. Heitweg B et al. Analytical Biochemistry (2003) also disclosed the use of the same small molecule substrate and its derivative for several recombinant HDAC isotypes in vitro. Wegener et al., Chemistry & Biology 10: 61-68 (2003) disclosed the use of fluorogenic HDAC substrates with an acetylated lysine, which upon deacetylation becomes a substrate for trypsin and then releases the fluorophore. Similarly, Biomol (Plymouth Meeting, Philadelphia) disclosed several fluorescent activity kits which could monitor HDAC activities in vitro (“HDAC Fluorescent Activity/Drug Discovery Kit”) or could specifically monitor SirT1, SirT2 or SirT3 activity in vitro. In vitro by using recombinant enzymes, inhibitory activity of suramin as well as activator activity of resverstrol could be monitored against Sirtuins and inhibitory activity of TSA could be monitored against HDACs in extracts or recombinant HDAC isotypes. Unfortunately, these and similar assays all require forming cellular extracts, which is time consuming and may result in artifacts from the extraction procedure.

The “HDAC Fluorescent Activity/Drug Discovery Kit” (Biomol) discloses an assay using cultured HeLa and Jurkat whole cells using an undisclosed acetylated HDAC (class I/II) pan-substrate that generates a fluorescent reporter molecule and measuring fluorescent HDAC cleavage product in the wells in which the cells were cultured. However, methods are lacking to measure 1) potency and isotype-specificity of a given class I/II HDAC inhibitor in whole cell context; 2) potency and isotype-specific of a Sirtuin inhibitors in whole cell context; and 3) HDAC activity from primary cells taken from a mammal or a mammal treated with HDAC class I/II inhibitors or sirtuin inhibitors. Especially in the latter senerio, primary whole cells taken from a mammal may not be susceptible to culturing and such cultured cells may not reflect the actual activity of HDAC in the cells in the body of the mammal.

There is therefore a need for assays which allow assessment of 1) isotype selectivity of an HDAC or sirtuin inhibitors in whole cell context and 2) level of protein deacetylase activity in whole cells taken directly from the body of the mammal.

### BRIEF SUMMARY OF THE INVENTION

The invention provides assays which allow assessment of the level of a protein deacetylase activity in primary intact whole cells taken directly from the body of the mammal or from bodily fluids.

In a first aspect, the invention provides a method for assessing total protein deacetylase activity of a protein deacetylase family or one or more member thereof in whole cells ex vivo. In the method according to this aspect of the invention whole cells from a mammal are provided and contacted with a cell-permeable pan-substrate for the protein deacetylase family or an isotype-specific substrate, wherein deacetylation of the substrate by the protein deacetylase family or the one or more member thereof generates a detectable reporter molecule. The quantity of the detectable reporter molecule is then measured. In preferred embodiments, the quantity of the detectable reporter molecule is measured against a control standard for the protein deacetylase family or the one or more member thereof.

In certain preferred embodiments, the protein deacetylase family is the histone deacetylase (HDAC) family. In certain preferred embodiments, the protein deacetylase family is the Sir2 family.

In a second aspect, the invention provides a method for assessing isotype-specific activity of one or more member of a protein deacetylase family from whole cells ex vivo, wherein one or more isotype of the protein deacetylase family provides a majority of the total deacetylase activity. In the method according to this aspect of the invention whole cells from a mammal are provided and contacted with a cell-permeable pan-substrate for the protein deacetylase family or a cell permeable isotype-specific substrate for the one or more member of the protein deacetylase family, wherein deacetylation of the substrate by the one or more protein deacetylase generates a detectable reporter molecule. A first aliquot of the cells is further contacted with an isotype-specific inhibitor of the one or more protein deacetylase that provides a majority of the total deacetylase activity and a second aliquot of the cells is not. The quantity of the detectable reporter molecule is then measured for the first and second aliquots and the quantity of protein deacetylase activity for each aliquot is compared. In preferred embodiments, the quantity of the detectable reporter molecule is measured against a control standard for the protein deacetylase family or the one or more member thereof.

In certain preferred embodiments, the protein deacetylase is a member of the histone deacetylase (HDAC) family. In certain preferred embodiments, the protein deacetylase is a member of the Sir2 family.

In a third aspect, the invention provides a method for assessing the activity of a specific isotype of one or more member of a protein deacetylase family ex vivo. In the method according to this aspect of the invention whole cells from a mammal are provided and contacted with a cell-permeable isotype-specific substrate for the one or more particular member of a protein deacetylase family, wherein deacetylation of the substrate by the protein deacetylase generates a detectable reporter molecule and measuring the quantity of the detectable reporter molecule.

In preferred embodiments, the quantity of the detectable reporter molecule is measured against a control standard for the protein deacetylase family or the one or more member thereof.

In certain preferred embodiments, the protein deacetylase is a member of the histone deacetylase (HDAC) family. In certain preferred embodiments, the protein deacetylase is a member of the Sir2 family.

In a fourth aspect, the invention provides a method for assessing the activity of a candidate pan-inhibitor of a protein deacetylase family or one or more member thereof in whole cells ex vivo. In the method according to this aspect of the invention whole cells from a mammal are provided and contacted with a cell-permeable pan-substrate for the protein deacetylase family or an isotype-specific substrate, wherein deacetylation of the substrate by the protein deacetylase family or one or more members thereof generates a detectable reporter molecule. A first aliquot of the cells is further contacted with a candidate pan-inhibitor of the protein deacetylase family and a second aliquot of the cells is not. The quantity of the detectable reporter molecule is then measured for the first and second aliquots and the quantity of protein deacetylase activity for each aliquot is compared. In preferred embodiments, the quantity of the detectable reporter molecule is measured against a control standard for the protein deacetylase family or the one or more members thereof.

In certain preferred embodiments, the protein deacetylase is a member of the histone deacetylase (HDAC) family. In certain preferred embodiments, the protein deacetylase is a member of the Sir2 family.

In a fifth aspect, the invention provides a method for assessing isotype-specific activity of a candidate inhibitor of a member of a protein deacetylase family from whole cells ex vivo, wherein one or more isotype of the protein deacetylase family provides a majority of the total deacetylase activity. In the method according to this aspect of the invention whole cells from a mammal are provided and contacted with a cell-permeable pan-substrate for the protein deacetylase family or a cell permeable isotype-specific substrate for the protein deacetylase family, wherein deacetylation of the substrate by the protein deacetylase generates a detectable reporter molecule. A first aliquot of the cells is further contacted with the candidate isotype-specific inhibitor of the protein deacetylase that provides a majority of the total deacetylase activity and a second aliquot of the cells is not. The quantity of the detectable reporter molecule is then measured for the first and second aliquots and the quantity of the detectable reporter molecule for each aliquot is compared. In preferred embodiments, the quantity of the detectable reporter molecule is measured against a control standard for the protein deacetylase family or the one or more member thereof.

In certain preferred embodiments, the protein deacetylase is a member of the histone deacetylase (HDAC) family. In certain preferred embodiments, the protein deacetylase is a member of the Sir2 family.

In a sixth aspect, the invention provides a method for assessing the efficacy of a pan-inhibitor of a protein deacetylase family or one or more member thereof in vivo. In the method according to this aspect of the invention, whole cells are provided from a mammal. The cells are contacted with a pan-substrate for the protein deacetylase family or an isotype specific substrate, wherein deacetylation of the substrate by the protein deacetylase family or one or more members thereof generates a detectable reporter molecule. The quantity of the reporter molecule is then determined. In preferred embodiments, the quantity is standardized against a known activity of the protein deacetylase family or the one or more members thereof. Next, the mammal is administered the pan-inhibitor. After an appropriate period of time, whole cells are again taken from the mammal and contacted with the pan-substrate. Next the quantity of the reporter molecule determined. In preferred embodiments, the quantity is standardized against a known activity of the protein deacetylase family or the one or more members thereof. Then the quantity of the reporter molecule after administration of the pan-inhibitor is compared with the quantity of the reporter molecule before administration before administration of the pan-inhibitor. Significant decrease in the quantity of the reporter molecule after administration of the pan-inhibitor is taken as a measure of efficacy.

In certain preferred embodiments, the protein deacetylase family is the histone deacetylase (HDAC) family. In certain preferred embodiments, the protein deacetylase family is the Sir2 family.

In a seventh aspect, the invention provides a method for assessing the efficacy and specificity of an isotype-specific inhibitor of a member of a protein deacetylase family in vivo. In the method according to this aspect of the invention, whole cells are provided from a mammal. The cells are contacted with an isotype-specific substrate for the one or more member of the protein deacetylase family, wherein deacetylation of the substrate by the protein deacetylase generates a detectable reporter molecule. The quantity of the reporter molecule is then determined. In preferred embodiments, the quantity is standardized against a known activity of the member of the protein deacetylase family. Next, the mammal is administered the isotype-specific inhibitor. After an appropriate period of time, whole cells are again taken from the mammal and contacted with the isotype-specific substrate. Next the quantity of the reporter molecule determined. In preferred embodiments, the quantity is standardized against a known activity of the one or more member of the protein deacetylase family. Then the quantity of the reporter molecule after administration of the isotype-specific inhibitor is compared with the quantity of the reporter molecule before administration of the isotype-specific inhibitor. Significant decrease in the quantity of the reporter molecule after administration of the isotype-specific inhibitor is taken as a measure of efficacy.

In certain preferred embodiments, the protein deacetylase is a member of the histone deacetylase (HDAC) family. In certain preferred embodiments, the protein deacetylase is a member of the Sir2 family.

In an eighth aspect, the invention provides a method for assessing the efficacy of a pan-inhibitor of total protein deacetylase family of mammals or one or more member thereof in vivo by measuring the quantity of a detectable reporter molecule in bodily fluids. In the method according to this aspect of the invention, the mammal is administered a cell-permeable pan-substrate for a protein deacetylase family or an isotype-specific substrate, wherein deacetylation of the pan-substrate or isotype-specific substrate generates a detectable reporter molecule. Bodily fluids from the mammal are obtained and the quantity of the detectable reporter molecule in the bodily fluids is determined. The mammal is then administered a pan-inhibitor of the protein deacetylase family and after an appropriate time period the mammal is administered the pan-substrate. Bodily fluids from the mammal are obtained and the quantity of the detectable reporter molecule in the bodily fluids is determined. The quantity of detectable reporter molecule in bodily fluids obtained prior to administration of the pan-inhibitor is then compared with the quantity of the detectable reporter molecule in bodily fluids after administration of the pan-inhibitor. Significant decrease in the quantity of the reporter molecule after administration of the pan-inhibitor is taken as a measure of efficacy.

In certain preferred embodiments, the protein deacetylase family is the histone deacetylase (HDAC) family. In certain preferred embodiments, the protein deacetylase family is the Sir2 family.

In a ninth aspect, the invention provides a method for assessing the efficacy of an isotype-specific inhibitor of one or more member of a protein deacetylase family in mammals in vivo by measuring the quantity of a detectable reporter molecule in bodily fluids. In the method according to this aspect of the invention, the mammal is administered a cell-permeable isotype-specific substrate for the one or more member of a protein deacetylase family, wherein deacetylation of the isotype-specific substrate generates the detectable reporter molecule. Bodily fluids from the mammal are obtained and the quantity of the detectable reporter molecule in the bodily fluids is determined. The mammal is then administered an isotype-specific inhibitor of one or more member of a protein deacetylase family and after an appropriate time period the mammal is administered the isotype-specific substrate. Bodily fluids from the mammal are obtained and the quantity of the detectable reporter molecule in the bodily fluids is determined. The quantity of detectable reporter molecule in bodily fluids obtained prior to administration of the isotype-specific inhibitor is then compared with the quantity of the detectable reporter molecule in bodily fluids after administration of the isotype-specific inhibitor. Significant decrease in the quantity of the reporter molecule after administration of the isotype-specific inhibitor is taken as a measure of efficacy. In certain preferred embodiments, the protein deacetylase family is the histone deacetylase (HDAC) family. In certain preferred embodiments, the protein deacetylase family is the Sir2 family.

In a tenth aspect, the invention provides a method for assessing the efficacy of a pan-activator of a protein deacetylase family or one or more member thereof in vivo. In the method according to this aspect of the invention, whole cells are provided from a mammal. The cells are contacted with a pan-substrate for the protein deacetylase family or an isotype specific substrate, wherein deacetylation of the substrate by the protein deacetylase family or one or more members thereof generates a detectable reporter molecule. The quantity of the reporter molecule is then determined. In preferred embodiments, the quantity is standardized against a known activity of the protein deacetylase family or the one or more members thereof. Next, the mammal is administered the pan-activator. After an appropriate period of time, whole cells are again taken from the mammal and contacted with the pan-substrate. Next the quantity of the reporter molecule determined. In preferred embodiments, the quantity is standardized against a known activity of the protein deacetylase family or the one or more members thereof. Then the quantity of the reporter molecule after administration of the pan-activator is compared with the quantity of the reporter molecule before administration before administration of the pan-activator. Significant increase in the quantity of the reporter molecule after administration of the pan-activator is taken as a measure of efficacy. In certain preferred embodiments, the protein deacetylase family is the histone deacetylase (HDAC) family. In certain preferred embodiments, the protein deacetylase family is the Sir2 family.

In an eleventh aspect, the invention provides a method for assessing the efficacy and specificity of an isotype-specific activator for of one or more member of a protein deacetylase family in vivo. In the method according to this aspect of the invention, whole cells are provided from a mammal. The cells are contacted with an isotype-specific substrate for the one or more member of the protein deacetylase family, wherein deacetylation of the substrate by the protein deacetylase generates a detectable reporter molecule. The quantity of the reporter molecule is then determined. In preferred embodiments, the quantity is standardized against a known activity of the member of the protein deacetylase family. Next, the mammal is administered the isotype-specific activator. After an appropriate period of time, whole cells are again taken from the mammal and contacted with the isotype-specific substrate. Next the quantity of the reporter molecule determined. In preferred embodiments, the quantity is standardized against a known activity of the one or more member of the protein deacetylase family. Then the quantity of the reporter molecule after administration of the isotype-specific activator is compared with the quantity of the reporter molecule before administration of the isotype-specific activator. Significant increase in the quantity of the reporter molecule after administration of the isotype-specific activator is taken as a measure of efficacy. In certain preferred embodiments, the protein deacetylase family is the histone deacetylase (HDAC) family. In certain preferred embodiments, the protein deacetylase family is the Sir2 family.

In a twelfth aspect, the invention provides a method for assessing the efficacy of a pan-activator of total protein deacetylase family of mammals or one or more members thereof in vivo by measuring the quantity of a detectable reporter molecule in bodily fluids. In the method according to this aspect of the invention, the mammal is administered a cell-permeable pan-substrate for a protein deacetylase family or one or more members thereof or an isotype specific substrate, wherein deacetylation of the pan-substrate or isotype-specific substrate generates the detectable reporter molecule. Bodily fluids from the mammal are obtained and the quantity of the detectable reporter molecule in the bodily fluids is determined. The mammal is then administered the pan-activator of the protein deacetylase family and after an appropriate time period the mammal is administered the pan-substrate or isotype specific substrate. Bodily fluids from the mammal are obtained and the quantity of the detectable reporter molecule in the bodily fluids is determined. The quantity of detectable reporter molecule in bodily fluids obtained prior to administration of the pan-activator is then compared with the quantity of the detectable reporter molecule in bodily fluids after administration of the pan-activator. Significant increase in the quantity of the reporter molecule after administration of the pan-activator is taken as a measure of efficacy. In certain preferred embodiments, the protein deacetylase is a member of the histone deacetylase (HDAC) family. In certain preferred embodiments, the protein deacetylase is a member of the Sir2 family.

In a thirteenth aspect, the invention provides a method for assessing the efficacy of an isotype-specific activator of one or more member of a protein deacetylase family in mammals in vivo by measuring the quantity of a detectable reporter molecule in bodily fluids. In the method according to this aspect of the invention, the mammal is administered a cell-permeable isotype-specific substrate for protein deacetylases, wherein deacetylation of the isotype-specific substrate generates the detectable reporter molecule. Bodily fluids from the mammal are obtained and the quantity of the detectable reporter molecule in the bodily fluids is determined. The mammal is then administered an isotype-specific activator of one or more member of a protein deacetylase family and after an appropriate time period the mammal is administered the isotype-specific substrate. Bodily fluids from the mammal are obtained and the quantity of the detectable reporter molecule in the bodily fluids is determined. The quantity of detectable reporter molecule in bodily fluids obtained prior to administration of the isotype-specific activator is then compared with the quantity of the detectable reporter molecule in bodily fluids after administration of the isotype-specific activator. Significant increase in the quantity of the reporter molecule after administration of the isotype-specific activator is taken as a measure of efficacy. In certain preferred embodiments, the protein deacetylase family is the histone deacetylase (HDAC) family. In certain preferred embodiments, the protein deacetylase family is the Sir2 family.

In a fourteenth aspect, the invention provides a method for assessing the activity of a candidate pan-activator of a protein deacetylase family or one or more members thereof in whole cells ex vivo. In the method according to this aspect of the invention whole cells from a mammal are provided and contacted with a cell-permeable pan-substrate for the protein deacetylase family or an isotype-specific substrate, wherein deacetylation of the substrate by the protein deacetylase family or one or more members thereof generates a detectable reporter molecule. A first aliquot of the cells is further contacted with a candidate pan-activator of the protein deacetylase family a second aliquot of the cells is not. The quantity of the detectable reporter molecule is then measured for the first and second aliquots and the quantity of protein deacetylase activity for each aliquot is compared. In preferred embodiments, the quantity of the detectable reporter molecule is measured against a control standard for the protein deacetylase family or the one or more members thereof.

In certain preferred embodiments, the protein deacetylase is a member of the histone deacetylase (HDAC) family. In certain preferred embodiments, the protein deacetylase is a member of the Sir2 family.

In a fifteenth aspect, the invention provides a method for assessing the activity of a candidate isotype-specific activator of a protein deacetylase family or one or more members thereof in whole cells ex vivo. In the method according to this aspect of the invention whole cells from a mammal are provided and contacted with a cell-permeable pan-substrate for the protein deacetylase family or an isotype-specific substrate, wherein deacetylation of the substrate by the protein deacetylase family or one or more members thereof generates a detectable reporter molecule. A first aliquot of the cells is further contacted with a candidate isotype-specific activator of one or more member of the protein deacetylase family and a second aliquot of the cells is not. The quantity of the detectable reporter molecule is then measured for the first and second aliquots and the quantity of protein deacetylase activity for each aliquot is compared. In preferred embodiments, the quantity of the detectable reporter molecule is measured against a control standard for the protein deacetylase family or the one or more members thereof.

In certain preferred embodiments, the protein deacetylase is a member of the histone deacetylase (HDAC) family. In certain preferred embodiments, the protein deacetylase is a member of the Sir2 family.

### BRIEF DESCRIPTION OF THE DRAWINGS

FIG. 1 shows intracellular and excellular HDAC activity in cultured 293T cells;

FIG. 2 shows a scheme for generation of a detectable reporter molecule for a representative cell permeable substrate.

FIG. 3 shows substrate preference of recombinant Sirt1, Sirt2 and Sirt3 toward three substrates

FIG. 4 shows whole cell HDAC activity as a function of cell numbers in cultured human cancer cells and normal cells.

FIG. 5 shows the effect of substrate concentration on HDAC whole cell activity in human cancer cell lines.

FIG. 6 shows inhibition of whole cell HDAC activity in human cancer cells by SAHA, Compound 2 and LAQ-824

FIG. 7a shows sirtuin-specific substrates are cell permeable and the effect of concentration of substrates on Sirtuin whole cell activity in human cancer cells.

FIG. 7b show that exogenous NAD+ has no effect on whole cell sirtuin activity in human cancer cells;

FIG. 8 shows suramin but not TSA can inhibit SirT1 activity in human cancer cells;

FIG. 9 shows resveratrol can activate SirT1 activity in human cancer cells;

FIG. 10 shows whole cell HDAC activity as a function of cell numbers in human white blood cells.

FIG. 11 shows dose-dependent inhibition of whole cell HDAC activity in human white blood cells by HDAC inhibitors (Compound 2 and LAQ-824); as well as their isotypic enzyme inhibitory activities.

FIG. 12 shows whole cell SirT1 activity in mouse blood from diabetic mice using SirT1 specific substrate;

FIG. 13 shows dose-dependent inhibition of whole cell Sirt1 activity in mouse white blood cells by sirtuin inhibitor suramin

FIG. 14 shows time-dependent inhibition of HDAC enzyme activity in white blood cells from mice treated with Compound 2

FIG. 15 shows dose-dependent inhibition of whole cell HDAC activity and histone acetylation in white blood cells from mice treated with Compound 2.

FIG. 16 shows dose-dependent antitumor activity of Compound 2 in A431 xenograft model in mice;

FIG. 17 shows whole cell HDAC activity in white blood cells from three healthy human volunteers and the processing error of this assay

FIG. 18 shows the time course of whole cell HDAC activity from three cancer patients treated with Compound 6 orally.

FIG. 19 shows the time course of plasma accumulation of Compound 6 in blood from three cancer patients treated with the HDAC inhibitor orally.

FIG. 20 shows the time course of induction of histone acetylation in white blood cells from three cancer patients treated with Compound 6 orally.

FIG. 21 shows whole cell HDAC activity of HCT116 cells as a function of cell number using a calorimetric assay.

FIG. 22 shows whole cell HDAC activity in 293T cells overexpressing either HDAC-1 or HDAC-6 and the expression level of HDAC-1 or HDAC-6 in these cells.

FIG. 23 shows detection of HDAC activity from serum isolated from mouse whole blood contacted with an HDAC substrate.

### DETAILED DESCRIPTION OF THE PREFERRED EMBODIMENTS

The invention relates to enzymatic assays for protein deacetylases. More particularly, the invention relates to such assays utilizing whole cells. The invention provides assays which allow assessment of the level of a protein deacetylase activity in whole cells taken directly from the body of a mammal or in bodily fluids.

In a first aspect, the invention provides a method for assessing total protein deacetylase activity of a protein deacetylase family or one or more members thereof in whole cells ex vivo. In the method according to this aspect of the invention whole cells from a mammal are provided and contacted with a cell-permeable pan-substrate for the protein deacetylase family or an isotype-specific substrate, wherein deacetylation of the substrate by the protein deacetylase family or the one or more members thereof generates a detectable reporter molecule. The quantity of the detectable reporter molecule is then measured. In preferred embodiments, the quantity of the detectable reporter molecule is measured against a control standard for the protein deacetylase family or the one or more members thereof.

In certain preferred embodiments, the protein deacetylase family is the histone deacetylase (HDAC) family. In certain preferred embodiments, the protein deacetylase family is the Sir2 family.

A “protein deacetylase family” is a group of related proteins having the ability to remove acetyl groups from basic side chains of amino acid residues of a protein. The term “mammal” specifically includes humans. “Whole cells” are intact cells, which may be present separately or as part of a tissue or a tumor. “Cell permeable pan-substrates” are molecules which penetrate cells and which do not provide a detectable reporter molecule in their native form, but which do provide a detectable molecule after cleavage by the members of the protein deacetylase family. A “cell permeable isotype-specific inhibitor” is a protein deacetylase inhibitor, or salt thereof, that inhibits one or more member, but less than all members of a protein deacetylase family. For example, compound 2 and the salt thereof (referred to herein as compound 6), described in the examples, are specific for HDAC-1, HDAC-2 and HDAC-3. A “detectable reporter molecule” is a molecule that provides a measurable signal in an assay. The nature of the molecule is not critical as long as it is measurable. Preferred detectable reporter molecules include, without limitation, colorometric molecules, fluorescent molecules, FRET-detectable molecules, enzymes, radiolabels and chemiluminescent molecules. A “protein deacetylase control standard” is a sample having a known level of protein deacetylase activity. The HDAC and Sir-2 families are those families that are known as such in the literature.

The whole cells can be contacted with a cell-permeable pan-substrate or isotype-specific inhibitor alone or in combination with a pharmaceutically acceptable carrier. As used herein, the term “pharmaceutically acceptable” refers to a material that does not interfere with the effectiveness of the assay and is compatible with a biological system such as a cell, tissue, or organism. As used herein, the term “carrier” encompasses any excipient, diluent, filler, salt, buffer, stabilizer, solubilizer, lipid, or other material well known in the art for use in pharmaceutical formulations. It will be understood that the characteristics of the carrier, excipient, diluent etc . . . , will depend on the route of administration for a particular application. The preparation of pharmaceutically acceptable formulations containing these materials is described in, e.g., Remington's Pharmaceutical Sciences, 18th Edition, ed. A. Gennaro, Mack Publishing Co., Easton, Pa., 1990.

In a second aspect, the invention provides a method for assessing isotype-specific activity of one or more member of a protein deacetylase family from whole cells ex vivo, wherein one or more isotype of the protein deacetylase family provides a majority of the total deacetylase activity. In the method according to this aspect of the invention whole cells from a mammal are provided and contacted with a cell-permeable pan-substrate for the protein deacetylase family or a cell permeable isotype-specific substrate for the one or more member of the protein deacetylase family, wherein deacetylation of the substrate by the one or more protein deacetylase generates a detectable reporter molecule. A first aliquot of the cells is further contacted with an isotype-specific inhibitor of the one or more protein deacetylase that provides a majority of the total deacetylase activity and a second aliquot of the cells is not. The quantity of the detectable reporter molecule is then measured for the first and second aliquots and the quantity of protein deacetylase activity for each aliquot is compared. In preferred embodiments, the quantity of the detectable reporter molecule is measured against a protein deacetylase control standard.

In certain preferred embodiments, the protein deacetylase is a member of the histone deacetylase (HDAC) family. In certain preferred embodiments, the protein deacetylase is a member of the Sir2 family.

An “isotype-specific activity” is a protein deacetylase activity that inhibits one or more member, but less than all members of a protein deacetylase family. For example, Compound 2 or Compound 6, described in the examples are specific for HDAC-1, HDAC-2 and HDAC-3. Certain other isotype-specific activities include inhibitors specific for a single member of a protein deacetylase activity, e.g., HDAC-1. One or more isotype may provide a majority of the total protein deacetylase either naturally, or because the cell has been transfected with the one or more isotype and overexpresses it. The terms “first aliquot” and “second aliquot” are used for convenience and do not imply which aliquot is prepared first temporally. All other definitions are as described above.

In a third aspect, the invention provides a method for assessing the activity of one or more specific isotype of a member of a protein deacetylase family. In the method according to this aspect of the invention, whole cells from a mammal are provided and contacted with a cell-permeable isotype-specific substrate for the one or more member of a protein deacetylase family, wherein deacetylation of the substrate by the protein deacetylase generates a detectable reporter molecule, and measuring the quantity of the detectable reporter molecule.

In preferred embodiments, the quantity of the detectable reporter molecule is measured against a protein deacetylase control standard.

In certain preferred embodiments, the protein deacetylase is a member of the histone deacetylase (HDAC) family. In certain preferred embodiments, the protein deacetylase is a member of the Sir2 family.

An “isotype-specific substrate” is a substrate for one or more member, but less than all members of a protein deacetylase family. Certain other isotype-specific substrates include substrates specific for a single member of a protein deacetylase activity, e.g., HDAC-1. All other definitions are as described above.

In a fourth aspect, the invention provides a method for assessing the activity of a candidate pan-inhibitor of a protein deacetylase family or one or more members thereof in whole cells ex vivo. In the method according to this aspect of the invention whole cells from a mammal are provided and contacted with a cell-permeable pan-substrate for the protein deacetylase family or an isotype specific substrate, wherein deacetylation of the substrate by the protein deacetylase family or the one or more members thereof generates a detectable reporter molecule. A first aliquot of the cells is further contacted with a candidate pan-inhibitor of the protein deacetylase family a second aliquot of the cells is not. The quantity of the detectable reporter molecule is then measured for the first and second aliquots and the quantity of detectable reporter molecule in each aliquot is compared. In preferred embodiments, the quantity of the detectable reporter molecule is measured against a control standard for the protein deacetylase or the one or more members thereof.

In certain preferred embodiments, the protein deacetylase is a member of the histone deacetylase (HDAC) family. In certain preferred embodiments, the protein deacetylase is a member of the Sir2 family.

A “candidate pan-inhibitor” is an inhibitor of protein deacetylase which is to be tested for its ability to inhibit all members of a protein deacetylase family. A “pan-substrate” is a substrate for all members of a protein deacetylase family. All other definitions are as described above.

In a fifth aspect, the invention provides a method for assessing isotype-specific activity of a candidate inhibitor of one or more member of a protein deacetylase family from whole cells ex vivo, wherein one or more isotype of the protein deacetylase family provides a majority of the total deacetylase activity. In the method according to this aspect of the invention whole cells from a mammal are provided and contacted with a cell permeable pan-inhibitor for the protein deacetylase family or a cell-permeable isotype-specific substrate for the protein deacetylase family, wherein deacetylation of the substrate by the protein deacetylase generates a detectable reporter molecule. A first aliquot of the cells is further contacted with the candidate isotype-specific inhibitor of the protein deacetylase that provides a majority of the total deacetylase activity and a second aliquot of the cells is not. The quantity of the detectable reporter molecule is then measured for the first and second aliquots and the quantity of the detectable reporter molecule for each aliquot is compared. In preferred embodiments, the quantity of the detectable reporter molecule is measured against a protein deacetylase control standard.

In certain preferred embodiments, the protein deacetylase is one or more member of the histone deacetylase (HDAC) family. In certain preferred embodiments, the protein deacetylase is a member of the Sir2 family.

“Isotype-specific activity of a candidate inhibitor” is a determination of whether an inhibitor of protein deacetylation is specific for one or more member, but less than all members of a protein deactylase family. All other definitions are as described above.

In a sixth aspect, the invention provides a method for assessing the efficacy of a pan-inhibitor of a protein deacetylase family or one or more members thereof in vivo. In the method according to this aspect of the invention, whole cells are provided from a mammal. The cells are contacted with a pan-substrate for the protein deacetylase family or an isotype-specific substrate, wherein deacetylation of the substrate by the protein deacetylase family or the one or more members thereof generates a detectable reporter molecule. The quantity of the reporter molecule is then determined. In preferred embodiments, the quantity is standardized against a known activity of the protein deacetylase family or the one or more members thereof. Next, the mammal is administered the pan-inhibitor. After an appropriate period of time, whole cells are again taken from the mammal and contacted with the pan-substrate or isotype-specific substrate. Next the quantity of the reporter molecule determined. In preferred embodiments, the quantity is standardized against a known activity of the protein deacetylase family or the one or more members thereof. Then the quantity of the reporter molecule after administration of the pan-inhibitor is compared with the quantity of the reporter molecule before administration of the pan-inhibitor. Significant decrease in the quantity of the reporter molecule after administration of the pan-inhibitor is taken as a measure of efficacy. In certain preferred embodiments the whole cells taken from the mammal prior to administration of the inhibitor are stored and the assays for pre-treatment levels of detectable reporter molecule and for post-treatment are performed simultaneously or nearly simultaneously.

In certain preferred embodiments, the protein deacetylase family is the histone deacetylase (HDAC) family. In certain preferred embodiments, the protein deacetylase family is the Sir2 family.

Administration of the pan-inhibitor may be by any acceptable route, including without limitation oral, parenteral, sublingual, intravenous, intraocular, topical, intranasal, intraventricular, intravesicular and intrarectal. Bodily fluids include, without limitation blood, plasma, sputum, urine and cerebrospinal fluid. In certain preferred embodiments, each quantitation of the detectable reporter molecule is standardized against a known activity of the protein deacetylase family. In certain preferred embodiments, the bodily fluid obtained before administration of the pan-inhibitor is saved and quantification of the detectable reporter molecule in bodily fluids obtained before and after administration may be done simultaneously or nearly simultaneously. All other definitions are as described above.

In a seventh aspect, the invention provides a method for assessing the efficacy of an isotype-specific inhibitor of one or more member of a protein deacetylase family in vivo. In the method according to this aspect of the invention, whole cells are provided from a mammal. The cells are contacted with an isotype-specific substrate for the member of the protein deacetylase family, wherein deacetylation of the substrate by the protein deacetylase generates a detectable reporter molecule. The quantity of the reporter molecule is then determined. In preferred embodiments, the quantity is standardized against a known activity of the member of the protein deacetylase family. Next, the mammal is administered the isotype-specific inhibitor. After an appropriate period of time, whole cells are again taken from the mammal and contacted with the isotype-specific substrate. Next the quantity of the reporter molecule determined. In preferred embodiments, the quantity is standardized against a known activity of the member of the protein deacetylase family. Then the quantity of the reporter molecule after administration of the isotype-specific inhibitor is compared with the quantity of the reporter molecule before administration of the isotype-specific inhibitor. Significant decrease in the quantity of the reporter molecule after administration of the isotype-specific inhibitor is taken as a measure of efficacy.

In certain preferred embodiments, the protein deacetylase is one or more member of the histone deacetylase (HDAC) family. In certain preferred embodiments, the protein deacetylase is one or more member of the Sir2 family.

Administration of the isotype-specific inhibitor may be by any acceptable route, including without limitation oral, parenteral, sublingual, intravenous, intraocular, topical, intranasal, intraventricular, intravesicular and intrarectal. Bodily fluids include, without limitation blood, plasma, sputum, urine and cerebrospinal fluid. In certain preferred embodiments, each quantitation of the detectable reporter molecule is standardized against a known activity of the protein deacetylase family. In certain preferred embodiments, the bodily fluid obtained before administration of the isotype-specific inhibitor is saved and quantification of the detectable reporter molecule in bodily fluids obtained before and after administration may be done simultaneously or nearly simultaneously.

All other definitions are as described above.

In an eighth aspect, the invention provides a method for assessing the efficacy of a pan-inhibitor of total activity of a protein deacetylase family in a mammal or one or more members thereof in vivo by measuring the quantity of a detectable reporter molecule in bodily fluids. In the method according to this aspect of the invention, the mammal is administered a cell-permeable pan-substrate for the protein deacetylase family or an isotype specific substrate, wherein deacetylation of the pan-substrate or isotype-specific substrate generates the detectable reporter molecule. Bodily fluids from the mammal are obtained and the quantity of the detectable reporter molecule in the bodily fluids is determined. The mammal is then administered a pan-inhibitor of the protein deacetylase family and after an appropriate time period the mammal is administered the pan-substrate or isotype-specific substrate. Bodily fluids from the mammal are obtained and the quantity of the detectable reporter molecule in the bodily fluids is determined. The quantity of detectable reporter molecule in bodily fluids obtained prior to administration of the pan-inhibitor is then compared with the quantity of the detectable reporter molecule in bodily fluids after administration of the pan-inhibitor. Significant decrease in the quantity of the reporter molecule after administration of the pan-inhibitor is taken as a measure of efficacy.

In certain preferred embodiments, the protein deacetylase family is the histone deacetylase (HDAC) family. In certain preferred embodiments, the protein deacetylase family is the Sir2 family.

Administration of the pan-substrate or isotype-specific substrate and the pan-inhibitor may be by any acceptable route, including without limitation oral, parenteral, sublingual, intravenous, intraocular, topical, intranasal, intraventricular, intravesicular and intrarectal. Bodily fluids include, without limitation blood, plasma, sputum, urine and cerebrospinal fluid. In certain preferred embodiments, each quantitation of the detectable reporter molecule is standardized against a known activity of the protein deacetylase family. In certain preferred embodiments, the bodily fluid obtained before administration of the pan-inhibitor is saved and quantification of the detectable reporter molecule in bodily fluids obtained before and after administration may be done simultaneously or nearly simultaneously.

All other definitions are as described above.

In a ninth aspect, the invention provides a method for assessing the efficacy of an isotype-specific inhibitor of one or more member of a protein deacetylase family in a mammal in vivo by measuring the quantity of a detectable reporter molecule in bodily fluids. In the method according to this aspect of the invention, the mammal is administered a cell-permeable isotype-specific substrate for the one or more member of the protein deacetylase family, wherein deacetylation of the isotype-specific substrate generates the detectable reporter molecule. Bodily fluids from the mammal are obtained and the quantity of the detectable reporter molecule in the bodily fluids is determined. The mammal is then administered an isotype-specific inhibitor of the one or more member of a protein deacetylase family and after an appropriate time period the mammal is administered the isotype-specific substrate. Bodily fluids from the mammal are obtained and the quantity of the detectable reporter molecule in the bodily fluids is determined. The quantity of detectable reporter molecule in bodily fluids obtained prior to administration of the isotype-specific inhibitor is then compared with the quantity of the detectable reporter molecule in bodily fluids after administration of the isotype-specific inhibitor. Significant decrease in the quantity of the reporter molecule after administration of the isotype-specific inhibitor is taken as a measure of efficacy.

In certain preferred embodiments, the protein deacetylase family is the histone deacetylase (HDAC) family. In certain preferred embodiments, the protein deacetylase family is the Sir2 family.

Administration of the isotype-specific substrate and the isotype-specific inhibitor may be by any acceptable route, including without limitation oral, parenteral, sublingual, intravenous, intraocular, topical, intranasal, intraventricular, intravesicular and intrarectal. Bodily fluids include, without limitation blood, plasma, sputum, urine and cerebrospinal fluid. In certain preferred embodiments, each quantitation of the detectable reporter molecule is standardized against a known activity of the protein deacetylase family. In certain preferred embodiments, the bodily fluid obtained before administration of the isotype-specific inhibitor is saved and quantification of the detectable reporter molecule in bodily fluids obtained before and after administration may be done simultaneously or nearly simultaneously. The detectable reporter molecule is capable of diffusing out of the cells and into bodily fluids.

All other definitions are as described above.

In a tenth aspect, the invention provides a method for assessing the efficacy of a pan-activator of a protein deacetylase family or one or more members thereof in vivo. In the method according to this aspect of the invention, whole cells are provided from a mammal. The cells are contacted with a pan-substrate for the protein deacetylase family or an isotype-specific substrate, wherein deacetylation of the substrate by the protein deacetylase family or one or more members thereof generates a detectable reporter molecule. The quantity of the reporter molecule is then determined. In preferred embodiments, the quantity is standardized against a known activity of the protein deacetylase family or the one or more members thereof. Next, the mammal is administered the pan-activator. After an appropriate period of time, whole cells are again taken from the mammal and contacted with the pan-substrate or isotype-specific substrate. Next the quantity of the reporter molecule determined. In preferred embodiments, the quantity is standardized against a known activity of the protein deacetylase family or the one or more members thereof. Then the quantity of the reporter molecule after administration of the pan-activator is compared with the quantity of the reporter molecule before administration before administration of the pan-activator. Significant increase in the quantity of the reporter molecule after administration of the pan-inhibitor is taken as a measure of efficacy. In certain preferred embodiments, the protein deacetylase family is the histone deacetylase (HDAC) family. In certain preferred embodiments, the protein deacetylase family is the Sir2 family. A pan-activator of a protein deacetylase family is a molecule that activates all members of the protein deacetylase family.

Administration of the pan-activator may be by any acceptable route, including without limitation oral, parenteral, sublingual, intravenous, intraocular, topical, intranasal, intraventricular, intravesicular and intrarectal. Bodily fluids include, without limitation blood, plasma, sputum, urine and cerebrospinal fluid. In certain preferred embodiments, each quantitation of the detectable reporter molecule is standardized against a known activity of the protein deacetylase family. In certain preferred embodiments, the bodily fluid obtained before administration of the pan-activator is saved and quantification of the detectable reporter molecule in bodily fluids obtained before and after administration may be done simultaneously or nearly simultaneously. The detectable reporter molecule is capable of diffusing out of the cells and into bodily fluids.

All other definitions are as described above.

In an eleventh aspect, the invention provides a method for assessing the efficacy and specificity of an isotype-specific actvator for of one or more member of a protein deacetylase family in vivo. In the method according to this aspect of the invention, whole cells are provided from a mammal. The cells are contacted with a cell permeable isotype-specific substrate for the one or more member of the protein deacetylase family, wherein deacetylation of the substrate by the protein deacetylase generates a detectable reporter molecule. The quantity of the reporter molecule is then determined. In preferred embodiments, the quantity is standardized against a known activity of the member of the protein deacetylase family. Next, the mammal is administered the isotype-specific activator. After an appropriate period of time, whole cells are again taken from the mammal and contacted with the isotype-specific substrate. Next the quantity of the reporter molecule determined. In preferred embodiments, the quantity is standardized against a known activity of the one or more member of the protein deacetylase family. Then the quantity of the reporter molecule after administration of the isotype-specific activator is compared with the quantity of the reporter molecule before administration of the isotype-specific inhibitor. Significant increase in the quantity of the reporter molecule after administration of the isotype-specific activator is taken as a measure of efficacy. In certain preferred embodiments, the protein deacetylase is a member of the histone deacetylase (HDAC) family. In certain preferred embodiments, the protein deacetylase is a member of the Sir2 family.

An isotype-specific activator of one or more member of a protein deacetylase family is a molecule that increases the activity and/or quantity of one or more member, but not all members of the protein deacetylase family. All other definitions are as described above.

Administration of the isotype-specific activator may be by any acceptable route, including without limitation oral, parenteral, sublingual, intravenous, intraocular, topical, intranasal, intraventricular, intravesicular and intrarectal. Bodily fluids include, without limitation blood, plasma, sputum, urine and cerebrospinal fluid. In certain preferred embodiments, each quantitation of the detectable reporter molecule is standardized against a known activity of the protein deacetylase family. In certain preferred embodiments, the bodily fluid obtained before administration of the isotype-specific activator is saved and quantification of the detectable reporter molecule in bodily fluids obtained before and after administration may be done simultaneously or nearly simultaneously. The detectable reporter molecule is capable of diffusing out of the cells and into bodily fluids.

All other definitions are as described above.

In a twelfth aspect, the invention provides a method for assessing the efficacy of a pan-activator of total protein deacetylase family of mammals or one or more members thereof in vivo by measuring the quantity of a detectable reporter molecule in bodily fluids. In the method according to this aspect of the invention, the mammal is administered a cell-permeable pan-substrate for a protein deacetylase family or an isotype-specific substrate, wherein deacetylation of the pan-substrate or isotype-specific substrate generates the detectable reporter molecule. Bodily fluids from the mammal are obtained and the quantity of the detectable reporter molecule in the bodily fluids is determined. The mammal is then administered the pan-activator of the protein deacetylase family and after an appropriate time period the mammal is administered the pan-substrate or isotype-specific substrate. Bodily fluids from the mammal are obtained and the quantity of the detectable reporter molecule in the bodily fluids is determined. The quantity of detectable reporter molecule in bodily fluids obtained prior to administration of the pan-activator is then compared with the quantity of the detectable reporter molecule in bodily fluids after administration of the pan-activator. Significant increase in the quantity of the reporter molecule after administration of the pan-activator is taken as a measure of efficacy. In certain preferred embodiments, the protein deacetylase is a member of the histone deacetylase (HDAC) family. In certain preferred embodiments, the protein deacetylase is a member of the Sir2 family.

All definitions are as described above.

In a thirteenth aspect, the invention provides a method for assessing the efficacy of an isotype-specific activator of one or more member of a protein deacetylase family in mammals in vivo by measuring the quantity of a detectable reporter molecule in bodily fluids. In the method according to this aspect of the invention, the mammal is administered a cell-permeable isotype-specific substrate for protein deacetylases, wherein deacetylation of the isotype-specific substrate generates the detectable reporter molecule. Bodily fluids from the mammal are obtained and the quantity of the detectable reporter molecule in the bodily fluids is determined. The mammal is then administered an isotype-specific activator of one or more member of a protein deacetylase family and after an appropriate time period the mammal is administered the isotype-specific substrate. Bodily fluids from the mammal are obtained and the quantity of the detectable reporter molecule in the bodily fluids is determined. The quantity of detectable reporter molecule in bodily fluids obtained prior to administration of the isotype-specific activator is then compared with the quantity of the detectable reporter molecule in bodily fluids after administration of the isotype-specific activator. Significant increase in the quantity of the reporter molecule after administration of the isotype-specific activator is taken as a measure of efficacy. In certain preferred embodiments, the protein deacetylase family is the histone deacetylase (HDAC) family. In certain preferred embodiments, the protein deacetylase family is the Sir2 family.

All definitions are as described above.

In a fourteenth aspect, the invention provides a method for assessing the activity of a candidate pan-activator of a protein deacetylase family in whole cells ex vivo. In the method according to this aspect of the invention whole cells from a mammal are provided and contacted with a cell-permeable pan-substrate for the protein deacetylase family or an isotype-specific substrate, wherein deacetylation of the substrate by the protein deacetylase family or one or more members thereof generates a detectable reporter molecule. A first aliquot of the cells is further contacted with a candidate pan-activator of the protein deacetylase family a second aliquot of the cells is not. The quantity of the detectable reporter molecule is then measured for the first and second aliquots and the quantity of protein deacetylase activity for each aliquot is compared.

In certain preferred embodiments, the protein deacetylase is a member of the histone deacetylase (HDAC) family. In certain preferred embodiments, the protein deacetylase is a member of the Sir2 family. In preferred embodiments, the quantity of the detectable reporter molecule is measured against a control standard for the protein deacetylase family or the one or more members thereof.

All definitions are as described above.

In a fifteenth aspect, the invention provides a method for assessing the activity of a candidate isotype-specific activator of a protein deacetylase family or one or more members thereof in whole cells ex vivo. In the method according to this aspect of the invention whole cells from a mammal are provided and contacted with a cell-permeable pan-substrate for the protein deacetylase family or an isotype-specific substrate, wherein deacetylation of the substrate by the protein deacetylase family or one or more members thereof generates a detectable reporter molecule. A first aliquot of the cells is further contacted with a candidate isotype-specific activator of one or more member of the protein deacetylase family and a second aliquot of the cells is not. The quantity of the detectable reporter molecule is then measured for the first and second aliquots and the quantity of protein deacetylase activity for each aliquot is compared. In preferred embodiments, the quantity of the detectable reporter molecule is measured against a control standard for the protein deacetylase family or the one or more members thereof.

In certain preferred embodiments, the protein deacetylase is a member of the histone deacetylase (HDAC) family. In certain preferred embodiments, the protein deacetylase is a member of the Sir2 family.

All definitions are as described above.

The following examples are intended to further illustrate certain particularly preferred embodiments of the invention and are not intended to limit the scope of the invention in any way.

### EXAMPLE 1

### Intracellular and Excellular Deacetylase Activity Of Human 293T Cells Using Boc-Lys(Ac)-AMC As Substrate

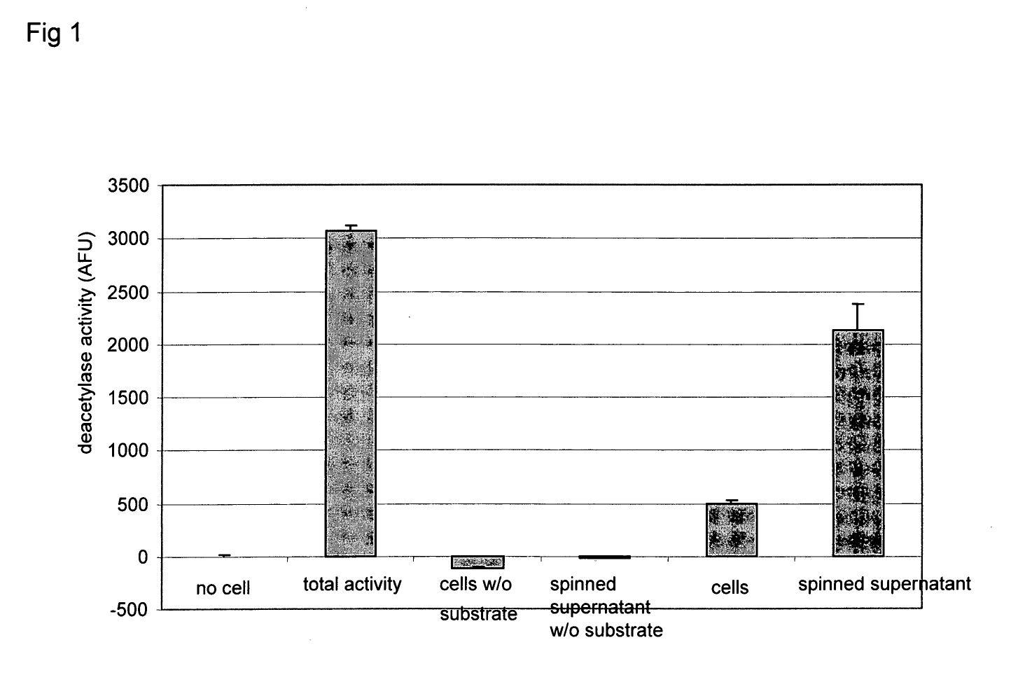

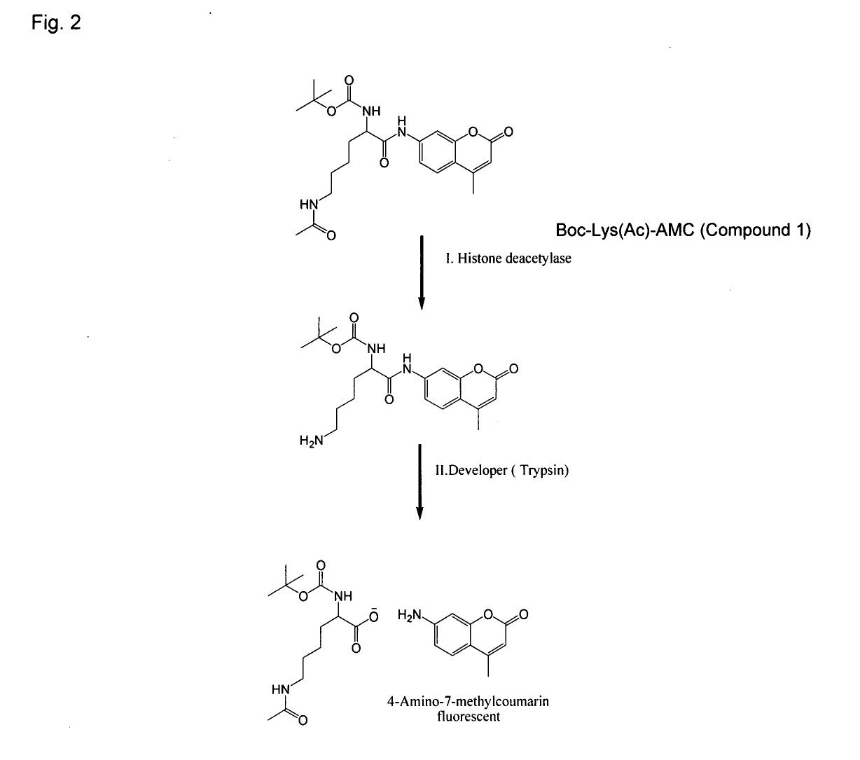

Freshly trypsinized cells (293T) were dispensed into 96-well black Costar E1A/RIA plates (Corning Inc., Corning, N.Y.). Small molecule substrate Boc-Lys(Ac)-AMC (Bachem Biosciences Inc., King of Prussia, Philadelphia) were added to cell suspension with the final concentration of 300 uM. Cells were placed in 37° C. incubator with 5% CO₂ for indicated time period. Supernatant was collected if necessary and subject to spinning. Reaction was stopped by adding a freshly prepared Fluor-de-Lys™ developer (Biomol, Plymouth Meeting, Philadelphia) with 1 uM TSA (Biomol, Plymouth Meeting, Philadelphia) in assay buffer (25 mM Tris, HCl pH8.0, 137 mM NaCl, 2.7 mM KCl, 1 mM MgCl2) plus 1% NP-40 into supernatant or cell pellets. Fluorescence was developed for 15 minutes at 37° C. and read in a fluorometer (SPECTRAMAX GeminiXS, Molecular Devices, Sunnylvale, Calif.) with an excitation wavelength at 360 nm, emission at 470 nm, and a cutoff of 435 nm. As shown in FIG. 1, significant intracellular and excellular deacetylase activity could be detected, suggesting that Boc-Lys(Ac)-AMC could permeablize into cells and generated product (Boc-Lys-AMC) could be diffused into culture media and could be subsequently detected by developing flurosecence. In contrast, when there is no substrates added, neither supernatant from cultured cells nor cell pellets do not have HDAC activity. The flow chart of the assay is shown in FIG. 2.

### EXAMPLE 2

### Substrate Boc-LysAc-AMC is not a Preferable Substrate for Situins In Vitro

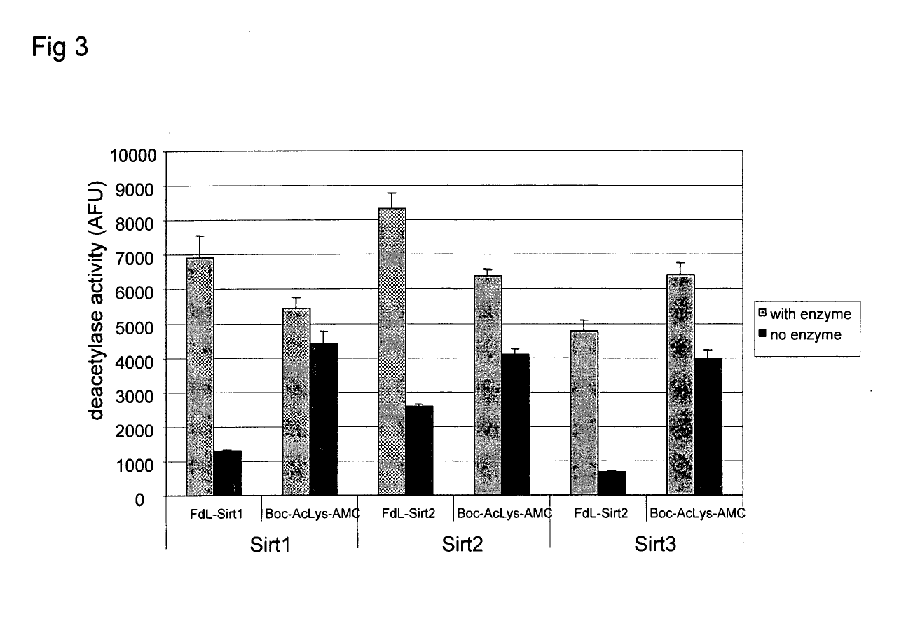

Human SirT1, 2, 3 recombinant enzymes were purchased from Biomol (Biomol, Plymouth Meeting, Philadelphia). Five units of each sirtuins were incubated with Fluor-de-Lys-SirT1 substrate (60 uM), Fluor-de-Lys-SirT2 substrate (200 uM for SirT2 and 30 uM for SirT3), or Boc-Lys(Ac)-AMC substrate (200 uM) in assay buffer (50 mM Tris-Cl, pH 8.0, 137 mM NaCl. 2.7 mM KCl, 1 mM MgCl2, 1 mg/ml BSA, 500 uM NAD⁺) for 45 minutes before the reaction is stopped and read, as suggested in Biomol user's manual or as described in Example 1. As shown in FIG. 3, Fluor-de-Lys-SirT1 substrate is a better substrate than Boc-Lys(Ac)-AMC toward recombinant Sirt1 enzyme, while Fluor-de-Lys-SirT2 substrate is a better substrate toward both Sirt2 and SirT3 enzymes.

### EXAMPLE 3

### Whole Cell Activity in Human Cancer Cells and Normal Cells Using Boc-Lys(Ac)-AMC

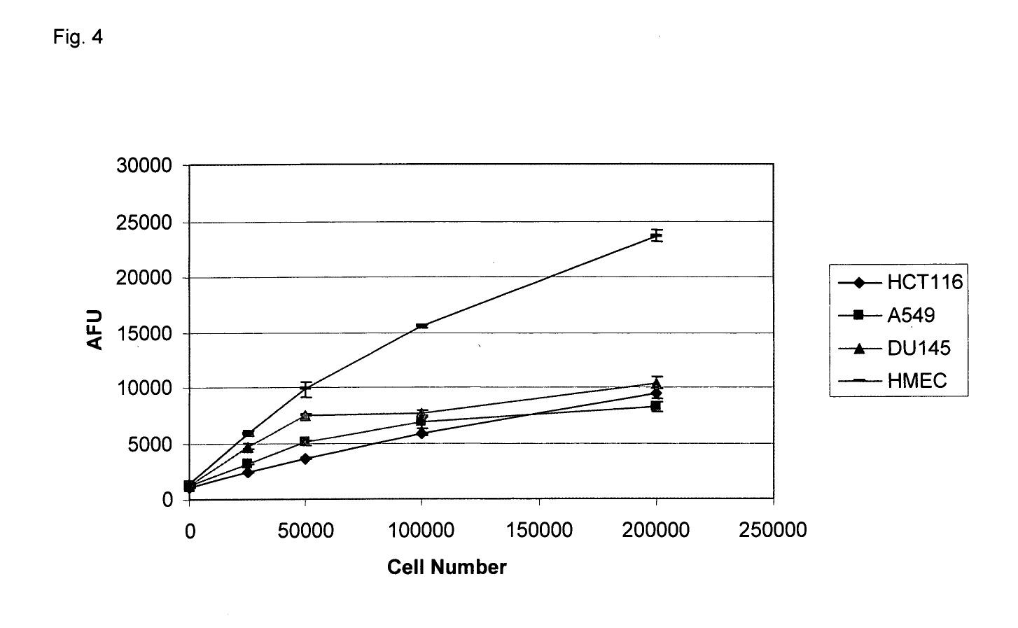

Freshly trypsinized cells were dispensed into 96-well black Costar E1A/RIA plates (Corning Inc., Corning, N.Y.). Small molecule substrate Boc-Lys(Ac)-AMC (Bachem Biosciences Inc., King of Prussia, Philadelphia) was added to cell suspension with the final concentration of 300 uM. Cells were placed in 37° C. incubator with 5% CO₂ for 90 minutes. Reaction was stopped by adding a freshly prepared Flouor-de-Lys™ deleveloper (Biomol, Plymouth Meeting, Philadelphia) with 1 uM TSA (Biomol) in assay buffer (25 mM Tris, HCl pH8.0, 137 mM NaCl, 2.7 mM KCl, 1 mM MgCl2) plus 1% NP-40. With the presence of 1% NP-40, both excellular and intracellular HDAC activity was measured in cultured cells altogether. Fluorescence was developed for 15 minutes at 37C and read in a fluorometer (SPECTRAMAX GeminiXS, Molecular Devices, Sunnylvale, Calif.) with an excitation wavelength at 360 nm, emission at 470 nm, and a cutoff of 435 nm. In cell lines we have tested (HCT116, A549, Du145, HMEC, 293T etc.), the total HDAC activity was a function of cell numbers (see FIG. 4).

### EXAMPLE 4

### Effect of Boc-LysAc-AMC Substrate Concentration on Deacetylase Activity in Human Cancer Cell Lines

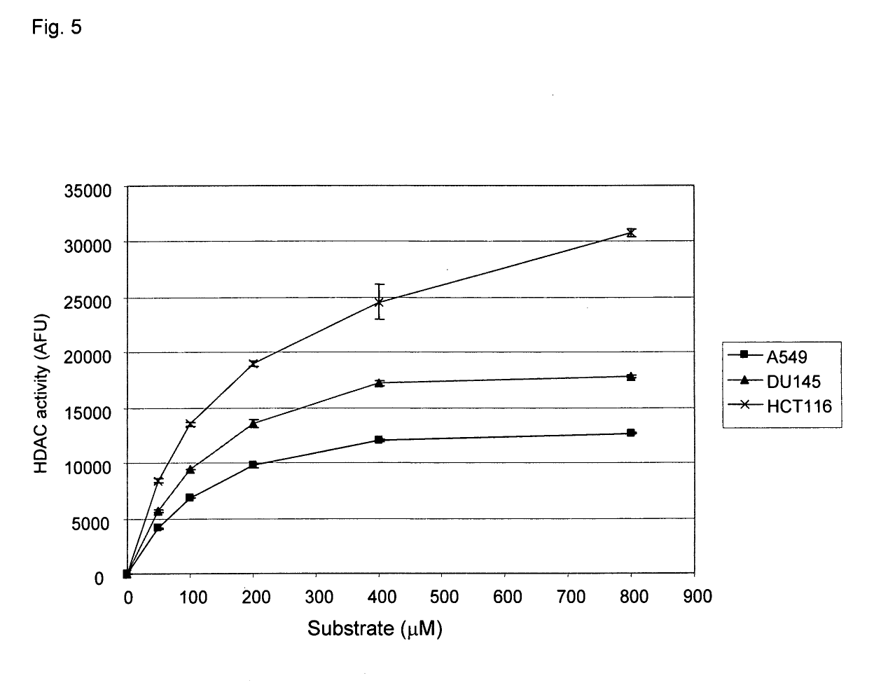

Cells were trypsinized and counted by trypan blue exclusion. Live cells (4×10⁴ A549 cells, or 1×10⁵ HCT116 cells, or 5×10⁴ Du145 cells) were distributed to each well of the 96-well plate. HDAC small molecule substrate Boc-Lys(Ac)-AMC with a range of final concentrations was added into cell suspensions and incubated with cells for 90 minutes at 37C before reaction was stopped, and fluorescence was developed and read. As shown in FIG. 5, effect of substrate concentration on whole cell deacetylase activity was measured. Km of Boc-Lys(Ac)-AMC ranged from 150 μM to 220 μM.

### EXAMPLE 5

### Activity of HDAC Pan or Isotype-Specific Inhibitors in Intact Cancer Cells Using Boc-Lys(Ac)-AMC as Substrate

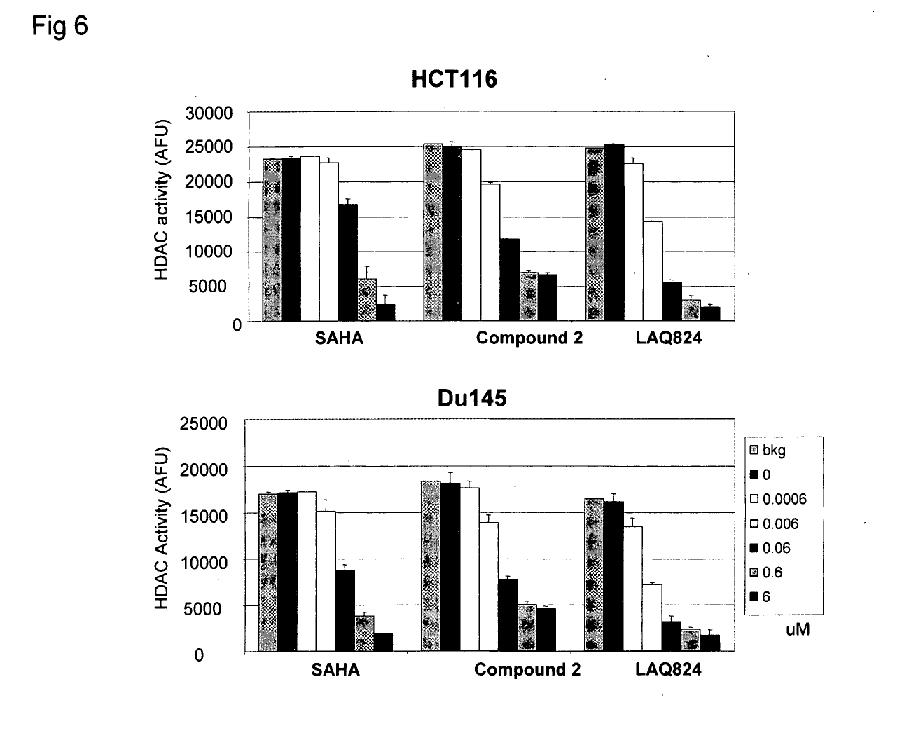

Human cancer cell lines (A549, Du145 and HCT116, 293T, Jurkat-T, Panc1) were treated with various concentrations of HDAC inhibitors for indicated time period before the enzyme substrate Boc-Lys(Ac)-AMC was added into cultured cells. Inhibitors could be pan-class I/II inhibitor (SAHA, LAQ-824) or isotype-specific class I inhibitors (against HD1, 2, 3), such as MS-275 or Compound 2. HDAC enzyme assay in intact cells was carried out as described in Example 3. The concentration which inhibits 50% of total HDAC activity (IC50) in whole cells was determined by analyzing the dose-response curve of enzyme inhibition, as shown in FIG. 6 and Table 1. However, also as shown in Table 1 below, in 293T cells while Compound 2 can inhibit HDAC activity in a dose-dependent manner, a CDK inhibitor (Compound 4 as described in Kim K S et. al., J Med. Chem. 45(18): 3905-3927 (2002).) or taxol has no effect on HDAC activity in whole cells using this assay.

TABLE 1
whole cell deacetylase IC50 of HDAC inhibitors or other
chemotherapeutic agents in various human cancer cells

| | IC50 (uM) | | | | | |
|---|---|---|---|---|---|---|
| | A549 | Du145 | HCT116 | 293T | Jurkat T | Panc-1 |
| Compound 2 | 0.4 | 0.6 | 0.4 | 0.5 | 0.2 | 0.2 |
| SAHA | 0.5 | 0.6 | 3 | 2 | 0.7 | 1 |
| MS-275 | 0.4 | 0.3 | 3 | 2 | 0.3 | 0.5 |
| LAQ-824 | 0.02 | 0.05 | 0.06 | 0.04 | 0.04 | nd |
| taxol | | | | >50 | | |
| compound 4 | | | | >50 | | |

results are mean IC50 from at least 2 independent experiments
cells were pre-incubated with inhibitors for 16 hours before reaction was stopped and read
compound 4 is a CDK2 inhibitor from BMS

### EXAMPLE 6

### Whole Cell Sirtuin Deacetylase Activity as a Function of Substrate Concentration in Human Cancer Cells Using Sirtuin Specific Substrate

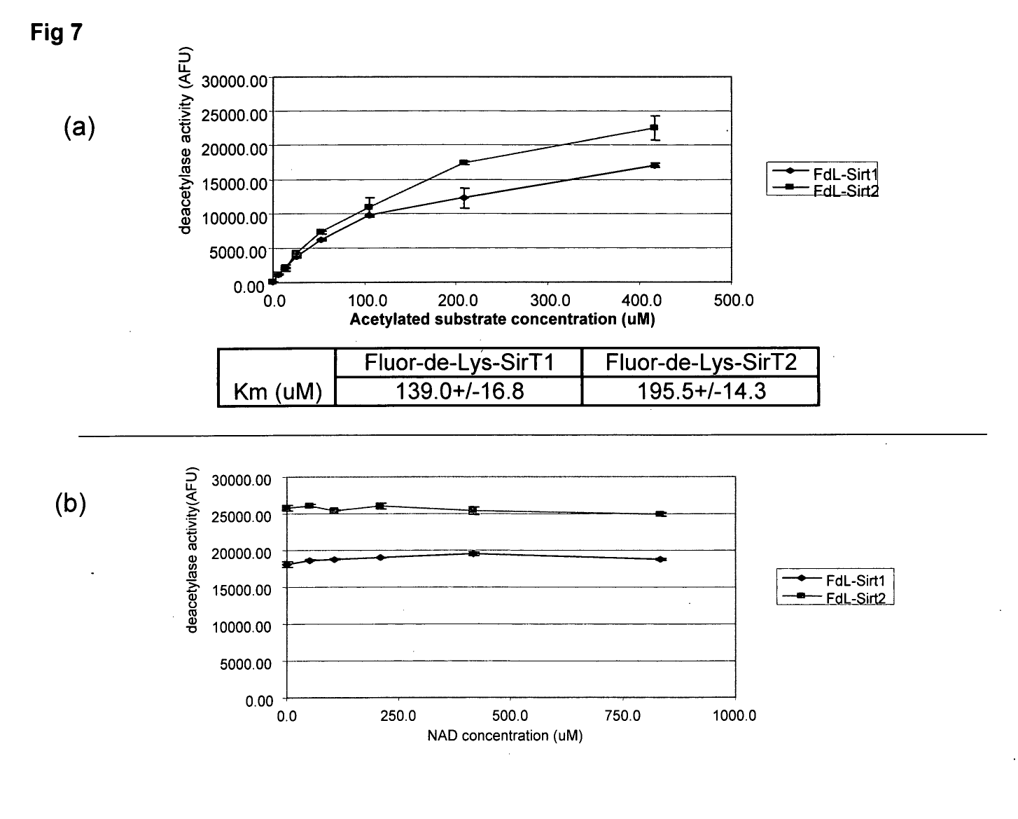

Cultured cells (HCT116) were trypsinized and counted by trypan blue exclusion. Live cells (2×10⁵) were distributed to each well of the 96-well plate. Sirtuin small molecule substrate Fluor-de-Lys-SirT1 substrate or Fluor-de-Lys-SirT2 substrate (Biomol, Plymouth Meeting, Philadelphia) with various concentrations were added to cultured cells and incubated for indicated time period before the reaction was stopped and read, as suggested in Biomol user's manual. We found that both Fluor-de-Lys-SirT1 or Fluor-de-Lys-SirT2 substrate (Biomol, Plymouth Meeting, Philadelphia) can be used as cell permeable substrates for sirtuins. Km of Fluor-de-Lys-SirT1 substrate or Fluor-de-Lys-SirT2 were determined in HCT116 cells to be 139.0 uM or 195.5 uM, respectively (FIG. 7a). Independently, we fixed concentrations of either Fluor-de-Lys-SirT1 substrate or Fluor-de-Lys-SirT2 in whole cells and analyzed the effect of exogeneous NAD+ concentration on whole cell sirtuin activity (FIG. 7b). We found that exogenous NAD+ had no effect on whole cell sirtuin activity, suggesting that NAD+ within cells is enough for whole cell sirtuin activity to reach its maxium. Unlike in vitro reactions using recombinant sirtuins, nicotinamide is ineffective at quenching the reaction and the fluorescent signal is being developed by cellular factors during incubation. Therefore the reading was done immediately after stopping.

### EXAMPLE 7

### Dose-Dependent Specific Inhibition of Sirtuin Enzyme Activity In Vitro by Suramin but not by TSA

Suramin in various concentrations was incubated with recombinant sirtuins 1-3 (Biomol, Plymouth Meeting, Philadelphia) for 45 minutes together with Sirtuin substrates (Fluor-de-Lys-SirT1 or Fluor-de-Lys-SirT2) in the set up as described in Example 2. Reaction is read as suggested in Biomol user's manual. As shown in Table 2, suramin inhibits Sirt1, SirT2, and SirT3 enzymes in vitro in a dose dependent manner. However, TSA up to 10 uM does not inhibit either Sirt1 or Sirt2 activity in vitro up to 10 uM.

TABLE 2
IC50 of suramin and TSA against recombinant Sirtuins

| | IC50 (uM) | | |
|---|---|---|---|
| | Sirt1 | Sirt2 | Sirt3 |
| (substrate) | FldSirt1 | FldSirt2 | FldSirt2 |
| suramin | 3 | 14 | 575 |
| TSA | >10 | >10 | nd |

nd: not determined

### EXAMPLE 8

### Suramin but not TSA Could Inhibit Whole Cell Sirtuin Activity in a Dose-Dependent Manner

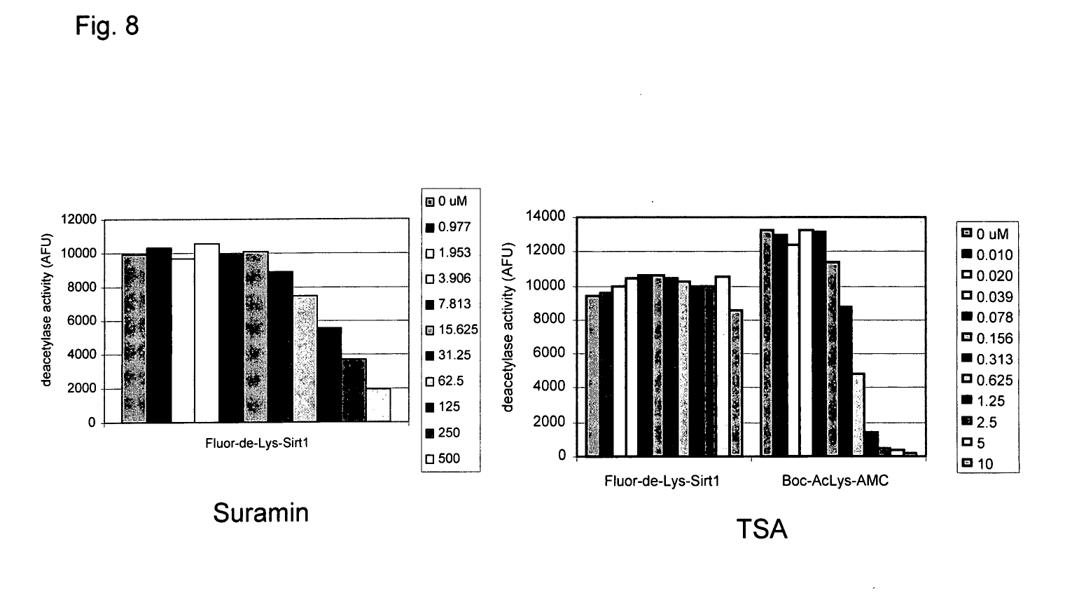

Cultured HCT116 cells were counted and distributed to each well of the 96-well plate. Suramin or TSA in various concentrations were incubated with cells for 1.5 hours before adding Sirtuin substrate (Fluor-de-Lys-SirT1, 500 uM) from Biomol (Plymouth Meeting, Philadelphia). After addition of Sirtuin substrate (Fluor-de-Lys-SirT1, 500 uM) or HDAC substrate Boc-Lys(Ac)-AMC (300 uM), the cells are further incubated for an additional 2 hours. Reaction was stopped and read as described in Examples 3 and 6. As shown in FIG. 8, Suramin but not TSA inihibits whole cell SirT1 activity in a dose-dependent manner. However, when Boc-Lys(AC)-AMC is used as substrates in whole cells, TSA could inhibit whole cell HDAC activity. This observation is consistent with our finding in Example 7 that TSA can not inhibit class III (Sirtuins) in vitro. Also in consistency with our observation in Example 3, we demonstrate that Boc-Lys(Ac)-AMC can be used to monitor class I plus class II HDAC activity in whole cells.

### EXAMPLE 9

### Dose-Dependent Specific Activation of Sirtuin Enzyme Activity In Vitro by Resveratrol

Resveratrol was incubated with recombinant sirtuins 1-3 (Biomol, Plymouth Meeting, Philadelphia) for 45 minutes together with Sirtuin substrates (Fluor-de-Lys-SirT1 or Fluor-de-Lys-SirT2). Reaction is read as described in Example 3. As shown in Table 3, resveratrol can activate Sirt1 enzymes in vitro in a dose dependent manner.

TABLE 3
EC50 (uM) of resveratrol to activate recombinant sirtuins in vitro

| | EC50 (uM) | | |
|---|---|---|---|
| | Sirt1 | Sirt2 | Sirt3 |
| (substrate) | FldSirt1 | FldSirt2 | FldSirt2 |
| Resveratrol | 45 | 912 | >2000 |

### EXAMPLE 10

### Dose-Dependent Specific Activation of Sirtuin Enzyme Activity in Human HCT116 Cells by Resveratrol

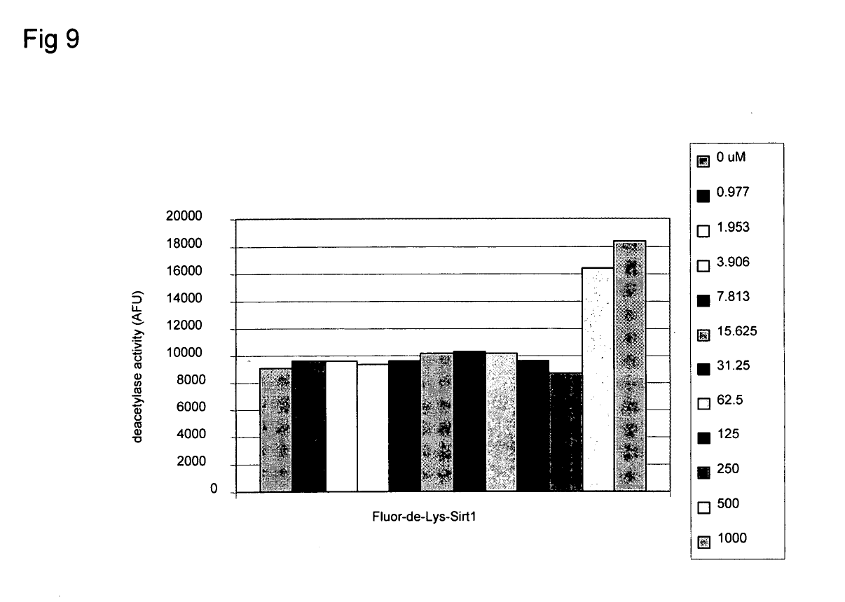

Human HCT116 cells were pre-incubated with resveratrol in various concentrations for 1.5 hour before Sirtuin substrate (Fluor-de-Lys-SirT1) was added and then further incubated for another 2 hours. Reaction is read and result is shown in FIG. 9. We demonstrate that resveratrol could activate whole cell sirtuin (SirT1) activity in a dose-dependent manner.

### EXAMPLE 11

### Whole Cell HDAC Activity of White Blood Cells Using Boc-Lys(Ac)-AMC as Substrate

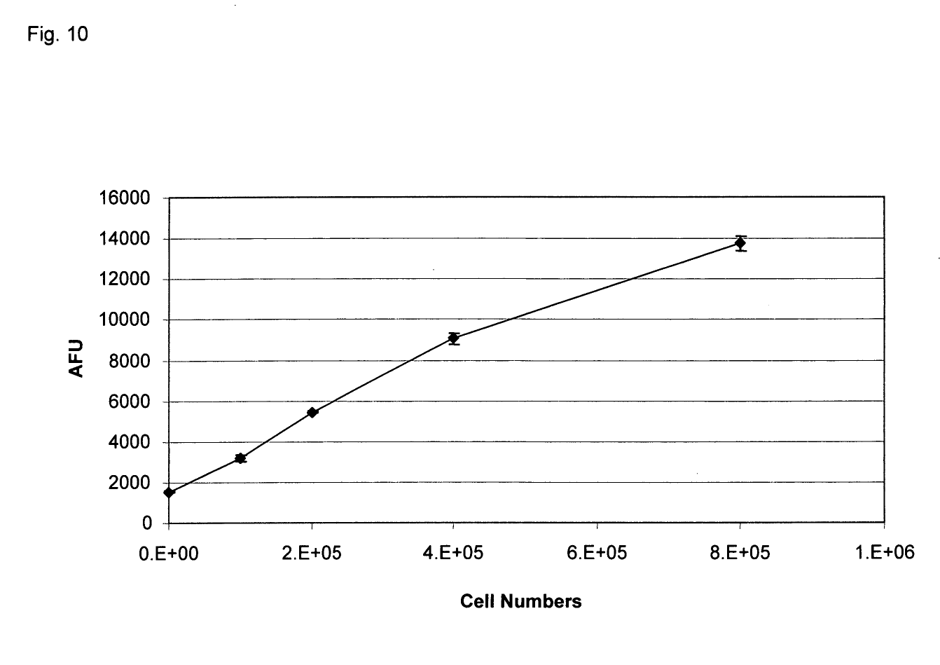

Whole blood (human or mouse) was centrifuged at 2500 rpm for 10 minutes at ambient temperature in a Sorvall RT-7 centrifuge (Mandel Scientific Co., Guelph, Ontario). Plasma was removed and buffy coat was collected. Five volumes of Erythrocyte Lysis Buffer (EL) (Qiagen Canada Inc., Mississauga, Ontario) were added to buffy coat. Buffy coat was incubated on ice for 20 minutes before it was centrifuged at 400 g for 10 minutes at 4° C. Supernatant was removed and buffy coat was washed twice with EL buffer and re-centrifugation. Buffy coat was resuspended in RPMI media and cells (white blood cells) were counted with trypan blue exclusion. White blood cells were plated into 96-well dish in RPMI plus 10% fetal bovine serum. HDAC small molecule substrate Boc-Lys(ac)-AMC was added to cell suspensions and incubated with cells for 90 minutes at 37° C. before reaction was stopped, and fluorescence was developed and read. As shown in FIG. 10, whole cell HDAC activity of human white blood cells was a function of cell numbers.

### EXAMPLE 12

### Ex Vivo Inhibition of Whole Cell HDAC Activity (Class I or Class II) in Human White Blood Cells Using Boc-LyAc-AMC as Substrate

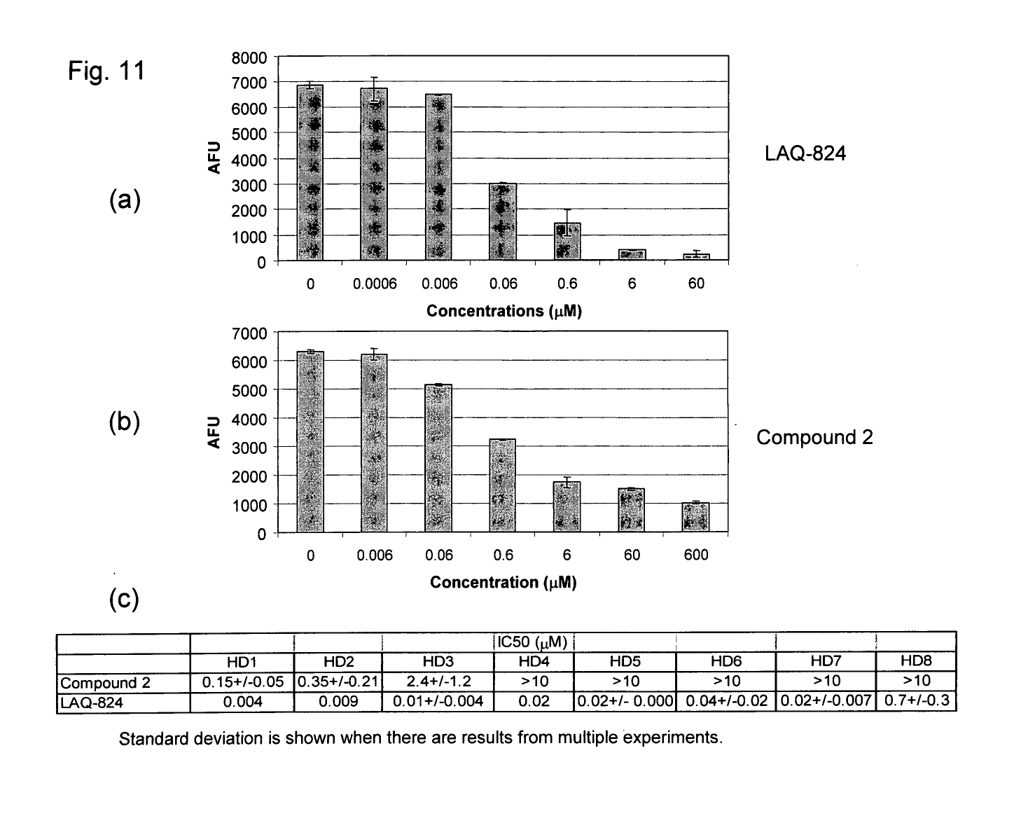

Human white blood cells (buffy coat) isolated from human donors were plated into 96-well dish in RPMI plus 10% fetal bovine serum. HDAC inhibitors with a range of dilutions were incubated with cells for 16 hours at 37 C with 5% CO₂. HDAC small molecule substrate Boc-Lys(ac)-AMC was added into cell suspensions and incubated with cells for 90 minutes before reaction was stopped, and fluorescence was developed and read. Both a pan-inhibitor (FIG. 11a; LAQ-824) and an isotype-specific inhibitor for HDACs1-3 (FIG. 11b; Compound 2) gave dose-dependent inhibition. FIG. 11c shows IC50s (in μM) of these inhibitors against recombinant HDAC enzymes in vitro using the same small molecule substrate Boc-Lys(Ac)-AMC. 70% of total HDAC activity was inhibited by the isotype-specific inhibitor, indicating that HDACs 1-3 provide most of the activity in white blood cells from human.

### EXAMPLE 13

### Whole Cell Sirtuin Activity Using White Blood Cells

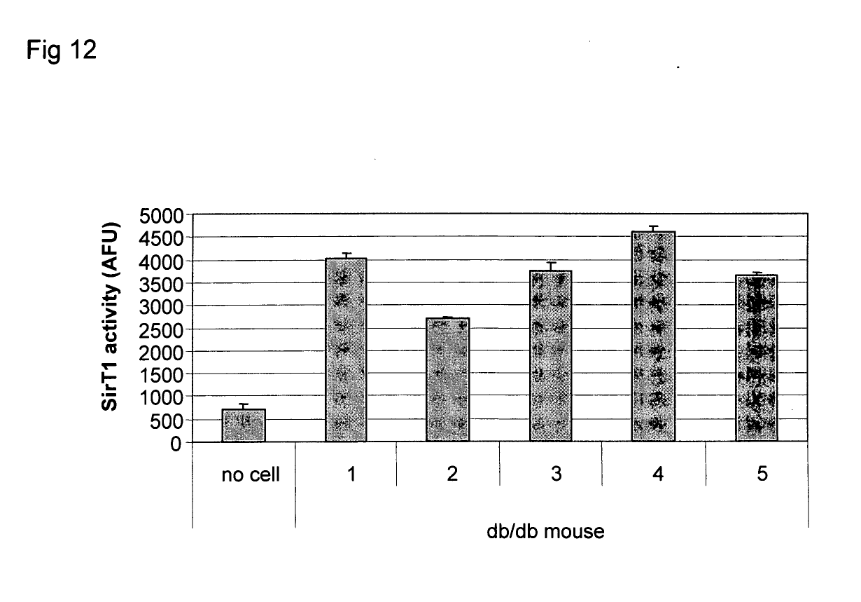

Blood from db/db mice (Jackson Laboratories, Bar Harbor, Me.) was collected in heparin tubes. Erythrocytes were lysed with five volumes of Erythrocyte Lysis buffer (EL, Qiagen Canada Inc, Mississauga, Ontario). White blood cells were recovered by centrifugation (400×g for 5 min), washed and resuspended in RPMI (+10% FBS), then counted. 4×10E5 cells were distributed in each well of a 96 well dish, together with 200 uM of Fluor-de-Lys Sirt1 (BioMol, Plymouth Meeting, Philadelphia), and incubated for 90 minutes. Reaction was stopped with one volume of assay buffer (50 mM Tris-Cl pH 8.0, 137 mM NaCl, 2.7 mM KCl, 1 mM MgCl₂) supplemented with 1× Developer II (BioMol, Plymouth Meeting, Philadelphia) and 1% NP40. In previous experiments, we had found that nicotinamide is ineffective at quenching the reaction, and that fluorescence is being developed by cellular factors during assay incubation, therefore the reading was done immediately after stopping. Results were shown in FIG. 12.

### EXAMPLE 14

### Ex Vivo Modulation of Whole Cell Sirtuin Activity in White Blood Cells Using Sirtuin Specific Substrate

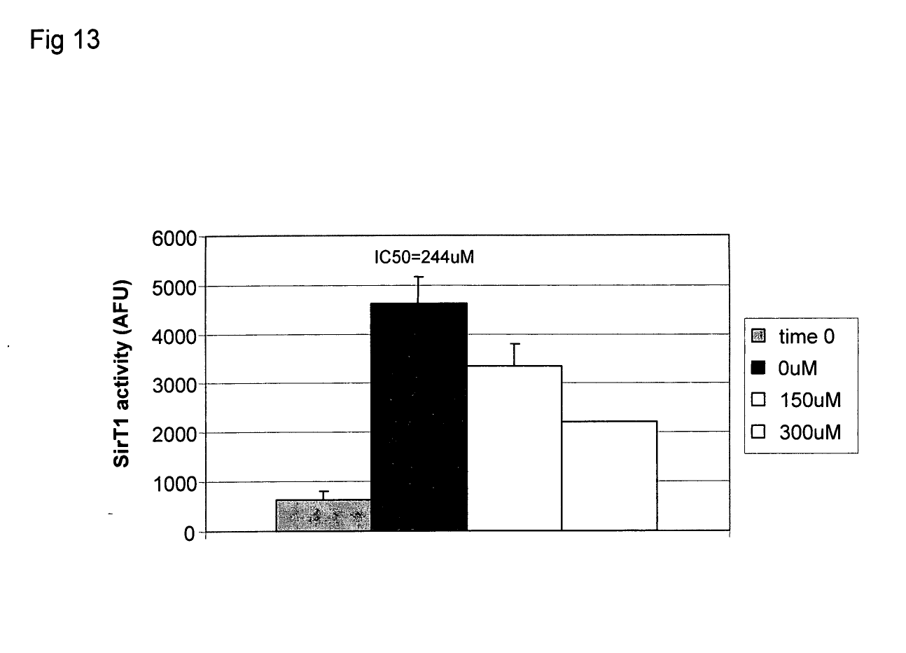

Mouse blood was collected in heparin tubes. Erythrocytes were lysed with five volumes of Erythrocyte Lysis buffer (EL, Qiagen Canada Inc, Mississauga, Ontario). White blood cells were recovered by centrifugation (400×g for 5 min), washed and resuspended in RPMI (+10% FBS), then counted. 4×10⁵ cells were distributed in each well of a 96 well dish and incubated with various doses of suramin, together with 200 uM of Fluor-de-Lys Sirt1 (BioMol, Plymouth Meeting, Philadelphia). After 90 minute incubation, reaction was stopped with one volume of assay buffer (50 mM Tris-Ci pH 8.0, 137 mM NaCl, 2.7 mM KCl, 1 mM MgCl₂) supplemented with 1× Developer II (BioMol, Plymouth Meeting, Philadelphia) and 1% NP40. As shown in FIG. 13, Suramin inhibits SirT1 enzymes in whole white blood cells.

### EXAMPLE 15

### Time-Dependent Inhibition of HDAC Activity in White Blood Cells in Animals Treated with Compound 2 In Vivo

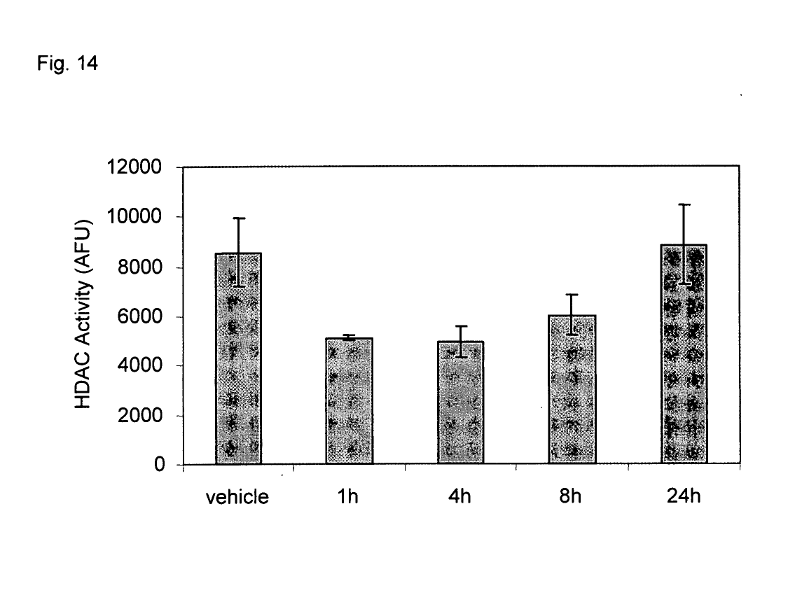

CD-1 mice (5 per group) were treated with either vehicle (PEG400:0.2N HCl in saline at 40:60 ratio) or Compound 2 at 90 mg/kg by oral administration for a single dose for indicated time period. Blood for each group of animals were arranged to harvest at the same point and were stored at 4 C overnight. White blood cells from individual animal were isolated. HDAC enzyme assay was performed using Boc-Lys (Ac)-AMC as described in Example 11. The results are shown in FIG. 14.

### EXAMPLE 16

### Plasma Concentration of an HDAC Inhibitor in Mice and in Human

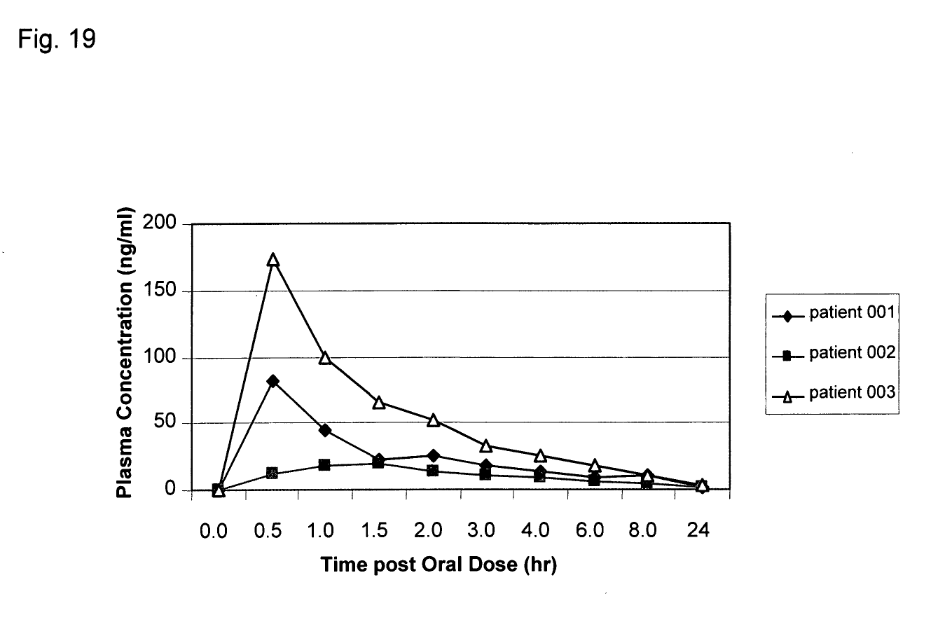

CD-1 mice (3 to 4 per group) were orally treated with a single dose of Compound 2 at 90 mg/kg. Blood was collected at indicated time points post dosing. Plasma concentration of Compound 2 in mouse blood was determined using HPLC-MS/MS. Assays were performed on an Agilent 1100 HPLC system (Agilent Technologies, Palo Alto, Calif., USA) coupled with an API2000 mass spectrometer (Applied Biosystems/MDS Sciex Concord, ON, Canada). ThermoHypersil 50×2.1 mm, 3 m, AQUASIL C18 column (Thermo Electron, WALTHAM, Mass., USA) was used. Mobile phase of A (water with 0.1% formic acid) and B (methanol with 0.1% formic acid) at an isocratic ratio of 45:55 were run at a flow rate of 300 mL/min. Sample injection was 5 ul. Positive multiple reaction monitoring (MRM) scan mode was utilized. Data was analyzed using Analyst™ program (Applied Biosystems/MDS Sciex Concord, ON, Canada). Proteins in mouse plasma were precipitated followed by evaporation to dryness, and were reconstituted with 0.1% formic acid aqueous solutions. As shown in Table 4, time course of plasma accumulation of Compound 2 correlates with that of HDAC enzyme inhibition by Compound 2 in mice (shown in FIG. 13). A similar method was used to detect plasma concentration of Compound 6 in patient in Phase I studies under a GLP condition in a contract research organization (FIG. 19).

TABLE 4
shows time-dependent accumulation of Compound 2 in
plasma from mice treated with Compound 2 orally in vivo

| Time post oral dose | mouse # | plasma concentration | average |
|---|---|---|---|
| 2 hour | 1 | 3.35 | |
| | 2 | 2.45 | |
| | 3 | 1.78 | |
| | 4 | 1.61 | |
| | | | 2.3 |
| 8 hour | 5 | 0.107 | |
| | 6 | 0.188 | |
| | 7 | 0.328 | |
| | | | 0.21 |
| 24 hour | 9 | 0.0263 | |
| | 10 | 0.0175 | |
| | 11 | 0.00972 | |
| | 12 | 0.00842 | |
| | | | 0.015 |

mice were single-dosed with Compound 2 at 90 mg/kg

### EXAMPLE 17

### Dose-Dependent Inhibition of Whole Cell HDAC Activity In Vivo

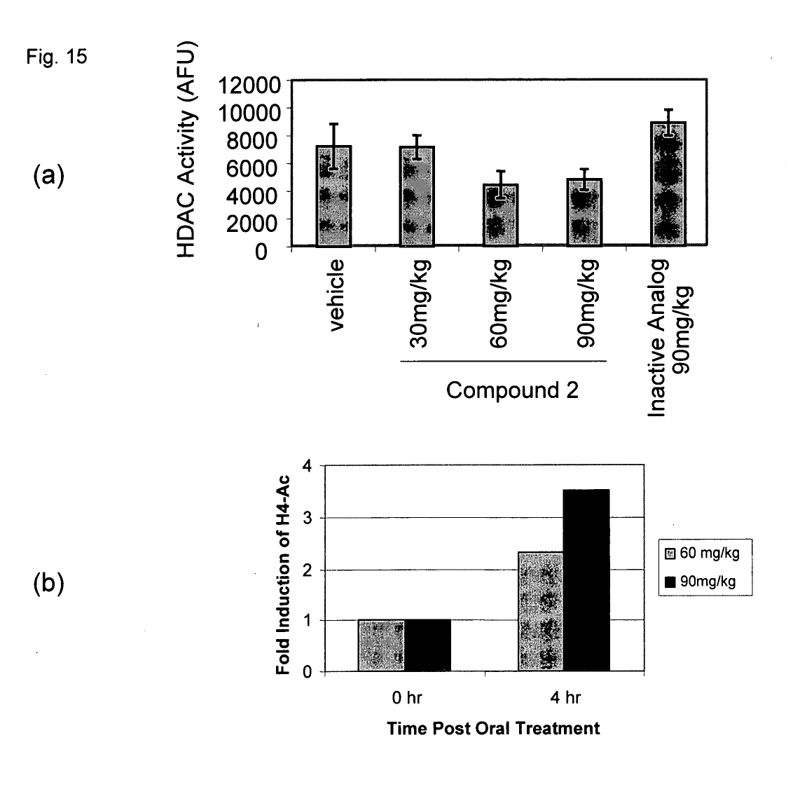

CD-1 mice (5 per group) were treated with either vehicle (PEG400:0.2N HCl in saline at 40:60 ratio) or Compound 2 or an inactive analog of Compound 2 (with similar molecular weight). Compounds were orally administered into mice at indicated single doses. Blood for each group of animals were harvested and stored at 4 C for overnight. White blood cells from individual animal were isolated. HDAC enzyme assay was performed using Boc-Lys (Ac)-AMC. Compound 2 but not its inactive analog inhibits HDAC activity in murine white blood cells in a dose-dependent manner (FIG. 15a).

### EXAMPLE 18

### Dose and Time-Dependent Induction of Histone Acetylation In Vivo

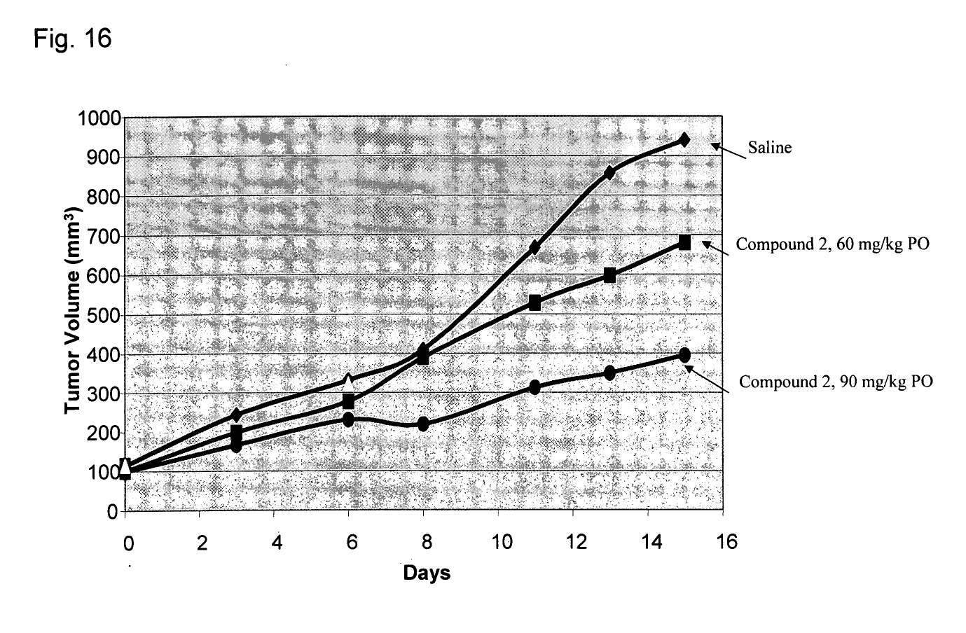

CD-1 nude mice (3 per group) were treated with either vehicle (PEG400:0.2N HCl in saline at 40:60 ratio) or Compound 2 (free base at 60 mg/kg or 90 mg/kg) by oral administration for 4 hours. Blood from each group were pooled and white blood cells were isolated. White blood cells (at least 2×10⁷) were lysed in ice-cold lysis buffer (10 mM Tris-HCl, pH 8.0, 1.5 mM MgCl2, 5 mM KCl, 0.5% NP-40, 12 uM DTT, 5 mM Sodium butyrate and freshly prepared protease inhibitors). Cells were incubated on ice for 10 minutes and centrifuged at 2000 rpm for 15 minutes at 4° C. in a IEC Micromax centrifuge (Fisher Scientific Ltd., Nepean, Ontario). Pellets were washed one time with cold lysis buffer and cold concentrated H₂SO₄ acid (final 0.4 M) was added to cell pellets, and resuspended pellets were incubated on ice for at least one hour before they were centrifuged at 15000 rpm for 5 minutes at 4° C. Supernatant was transferred to a 15 ml polypropylene Falcon tube (Becton Dickinson Laboratories, Franklin Lakes, N.J.) and acetone (10× volumes of the supernatant) was added. Supernatant with acetone was incubated at −20° C. for overnight and histones were recovered by centrifugation at 2000 rpm for 5 minutes at 4° C. Acid-extracted histones were air dried and resuspended in water and protein concentration determined by using BioRad protein assay (Bio-Rad Laboratories (Canada) Ltd., Mississauga, Ontario). Histones from white blood cells were analyzed by SDS-PAGE followed by Western blot using anti-acetylated H4 histone or anti-histone H4 antibodies. Acetylation of H4 histone for each group were normalized against that of vehicle-treated group. Enzyme inhibition of HDAC activity by Compound 2 in blood correlated with its induction of histone acetylation (FIG. 15b). Interestingly, the dose where Compound 2 can inhibit 50% of enzyme activity in white blood cells (60 mg/kg) is approximately the dose which leads to significant anti-tumor activity in vivo (FIG. 16). Enzyme inhibition of HDAC by Compound 2 correlated with its antitumor activity in mice (see below).

### EXAMPLE 19

### Dose-Dependent Antitumor Activity of Compound 2 in A431 Human Epidermoid Carcinoma Xenograft Model in Nude Mice

CD-1 Nude Mice bearing human A431 tumors (8 per group) were treated with either saline alone or various doses of Compound 2 in PEG400:0.2N HCl in saline at 40:60 ratio daily by oral administration. Briefly, A431 cells (2 million) were injected subcutaneously in the animal flank and allowed to form solid tumors. Tumor fragments were passaged in nude mice for a minimum of three times before their use. Tumor fragments (about 30 mg) were implanted subcutaneously through a small surgical incision under general anesthesia to CD1 female nude mice (6-8 weeks old, from Charles River Laboratories, Wilmington, Mass.). Recipient animals were treated with saline or HDAC inhibitors by oral administrations when the tumor sizes reached about 100 mm³. Tumor volumes and gross body weight of animals were monitored twice weekly for up to 2 weeks. Each experimental group contained at least 8 animals. Student's Tests were used to analyze the statistical significance between numbers in data sets. Tumor volumes were monitored for 2 weeks. The results are shown in FIG. 16.

### EXAMPLE 20

### Whole Cell HDAC Activity in White Blood Cells from Healthy Volunteers

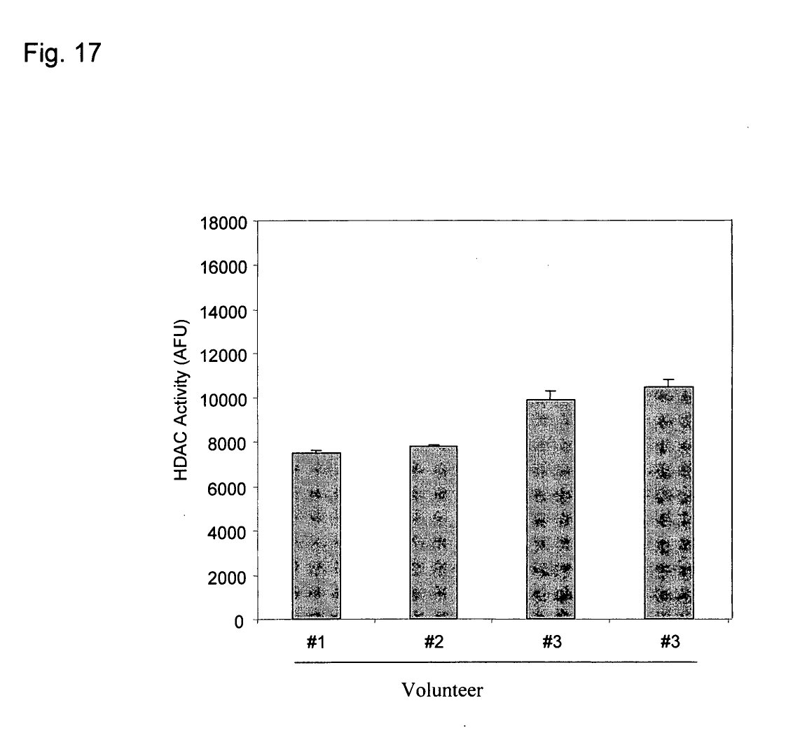

Freshly drawn human blood from three volunteers were analyzed. HDAC activity in isolated white blood cells (800,000 cells) was analyzed using Boc-Lys(AC)-AMC, as described in Example 11. Two independent analyses were done using white blood cells from volunteer #3 to show the processing error of this method. The processing error was very small (see FIG. 17).

### EXAMPLE 21

### Time Course of Whole Cell HDAC Activity and Histone Acetylation from Patients Treated with Compound 6

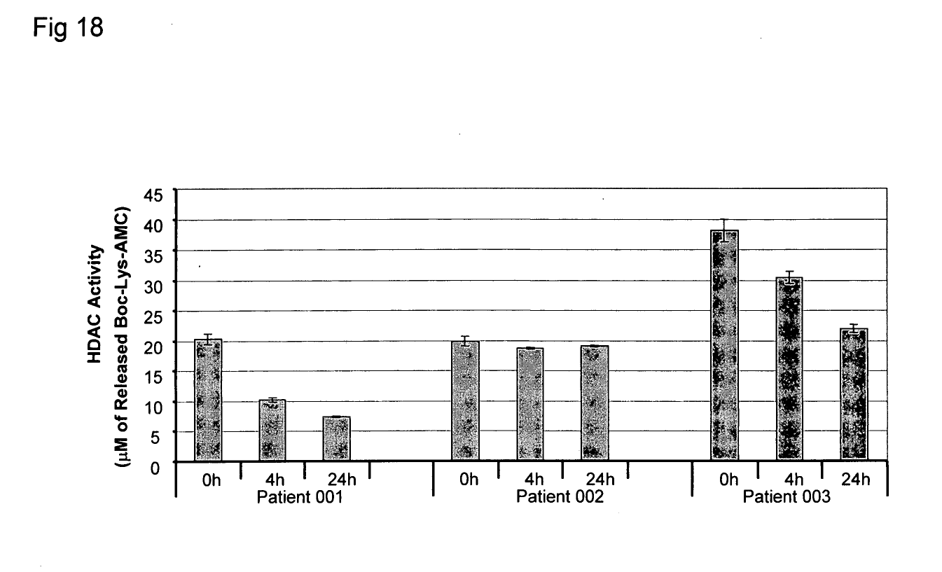

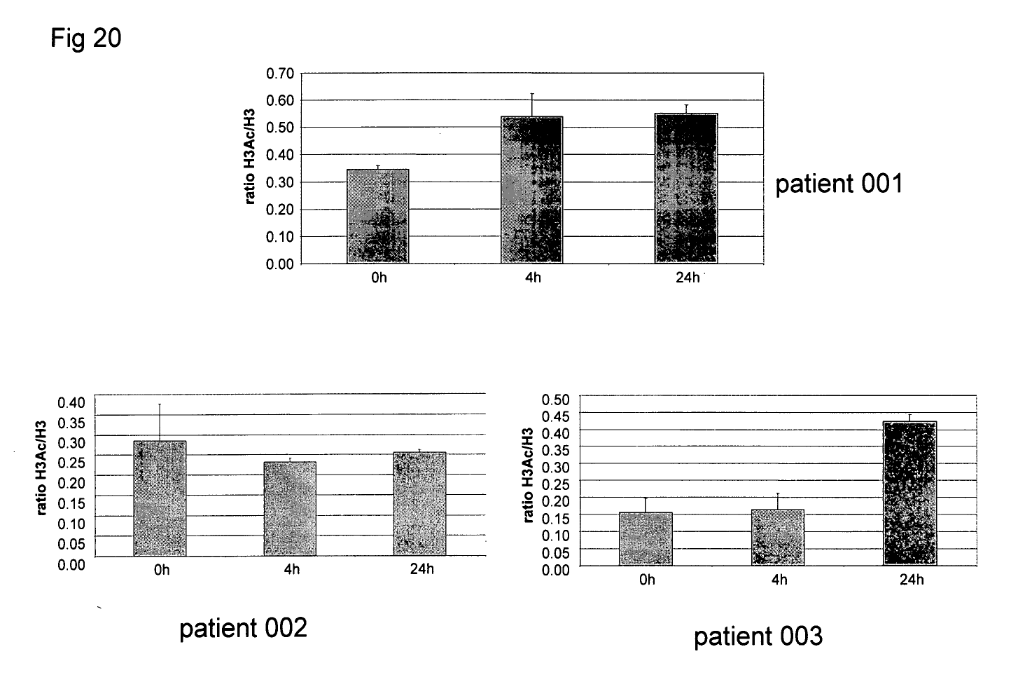

Three patients were treated with Compound 6 (12 mg/m²) at time 0 hour, blood samples were retrieved from patients at indicated time points post treatment. Whole cell HDAC activity of isolated white blood cells (800,000 cells) was analyzed using Boc-Lys(AC)-AMC, as described in Example 11. In FIG. 18, time course of HDAC enzyme inhibition in white blood cells in three individual patients is shown. HDAC enzyme inhibition by Compound 6 correlated with pharmacokinetics of Compound 6 in blood of these patients. Time-dependence of the plasma concentrations of Compound 6 in these three patients are shown in FIG. 19. Patient #001 and #003, who had a better accumulated drug exposure in the blood for the first 24 hours post treatment, exhibited more dramatic reduction on HDAC enzyme activity in white blood cells than Patient #002, who had much less drug exposure during the same period. We further demonstrated that enzyme inhibition in white blood cells from patients correlated well with induction of histone acetylation by Compound 6. In FIG. 20, time-dependence of induction of histone acetylation of patients was analyzed. Patients were treated with Compound 6 (12 mg/m 2) at time 0 hour, blood samples were retrieved from patients at indicated time points post treatment. White blood cells were isolated and histones were extracted as shown in Example 18. Histone H3 acetylation was analyzed using ELISA on isolated histones. (see blow). Inhibition of whole cell enzyme activity by Compound 6 (FIG. 18) in white blood cells from patients treated with Compound 6 in vivo correlated well with induction of histone acetylation (FIG. 20) as well as the plasma accumulation of Compound 6 (FIG. 19).

### EXAMPLE 22

### Sandwich ELISA on Purified Histone to Detect Histone Acetylation

Isolated histones (6 ug) from white blood cells (as described in Example 18) of patients treated with Compound 6 in vivo was used to analyze histone acetylation. Briefly, anti-histone (H11-4) antibody (Roche, Laval, Quebec) at 1 ug/ml was used to coat a black plate (Nunc437111 plates, VWR, Ville Mont-Royal, Quebec) at 22° C. for 2 hours. Coated plates were washed twice in PBS and were blocked with (0.1% TritonX-100 and 1% bovine serum albumin in PBS) at 22° C. for 40 minutes. Primary antibody, which is either rabbit polyclonal anti-acetyl-H3 (Upstate, Waltham, Mass.) antibody at 1:500 dilution or rabbit polyclonal anti-H3 antibody (Abcam, Cambridge, Mass.) at 1:2500 dilution, was used together with isolated histones (6 ug in blocking solution). Plates were incubated with primary antibody and histones for 45 minutes at 22° C. and washed three times subsequently using blocking solution (see above). Secondary antibody, which is goat polyclonal anti-rabbit-HRP antibody (Sigma, St-Louis, Mo.) in 1:8000 dilution in blocking solution, was used to incubate for 45 minutes at 22° C. Plates were washed subsequently twice with blocking buffer and twice with PBS. Reaction was developed by adding 50 uM Amplex-Red (Invitrogen Canada Inc., Burlington, Ontario) plus 200 uM H₂O₂ and incubate for 30 minutes in the dark. Fluroscence is read on the fluorometer (SpectraMax GeminiXS, Molecular Devices) at excitation wavelength at 550 nm and emission wavelength at 610 nm, with a cut-off of 590 nm. Histone acetylation of isolated histones from three patients treated with Compound 6 is shown in FIG. 20.

### EXAMPLE 23

### Whole Cell HDAC Activity in Human Cancer Cells Measured by a Colormetric Assay Using Cell-Permeable Substrates

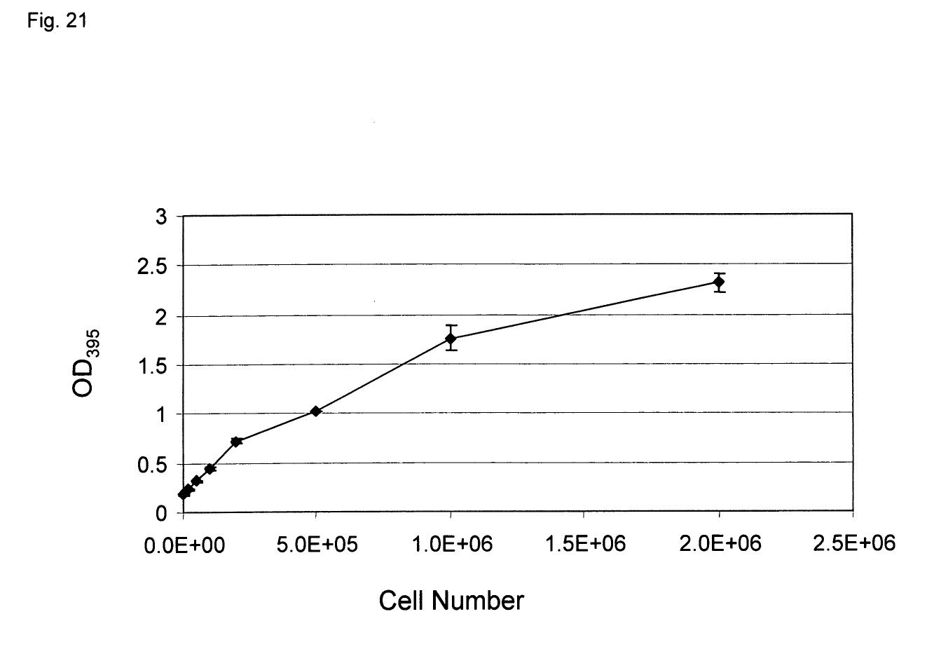

HCT116 cells were trypsinized and counted. Cells were plated in 96-well Costar black plates (E1A/RIA) in their growth medium and whole cell HDAC enzyme assay was done using “Colorimetric HDAC activity assay kit” from Biovision (Mountain View, Calif.). HDAC colorimetric substrates (Boc-Lys(Ac)-pNA) were added into cell suspensions at a final concentration of 300 uM. Plates were incubated for 90 minutes at 37 C with 5% CO₂. Before the reaction was stopped, read OD at 405 nm to get a background. Reaction was stopped by adding “Lysine developer” (from the kit) and plates incubated at 37 C for 30 minutes before O.D. was read at 405 nM on a SpectraMax 190 (Molecular Devices, Sunnylvale, Calif.). In FIG. 21, we demonstrate that whole cell HDAC activity as a function of cell number when using Boc-Lys(Ac)-pNA as substrate. Therefore, whole cell HDAC activity can be measured using a cell-permeable substrate no matter what kind of receptor molecule is generated from the substrate by HDAC enzymes.

### EXAMPLE 24

### Monitoring Isotype-Specificity and Potency of HDAC Inhibitors in Cells Predominantly Overexpressing HDAC-1 or HDAC-6 Isotypes

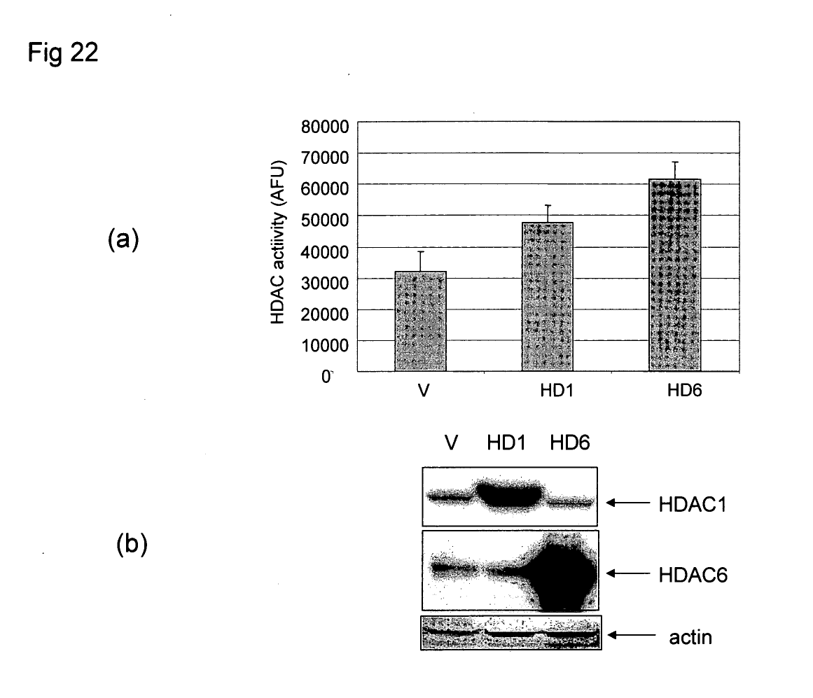

293T cells were infected with lentivirus encoding human HDAC-1 or HDAC-6. Cells were selected against puromycin to get antibiotic-resistant populations. Cells were plated in a 96-well plate and incubated with a small molecule substrate (Boc-Lys(Ac)-AMC) before reaction was stopped and read. Expression level of HDAC-1 or HDAC-6 in these cells were analyzed by immunoblotting. As shown in FIG. 22a, overexpression of HDAC-1 or HDAC-6 in 293T cells significantly increases overall HDAC activity and overexpression of HDAC-1 or HDAC-6 in 293T cells was confirmed by Western blot (FIG. 22b). We profile isotype selectivity and potency of several HDAC inhibitors using these cell lines, as shown in Table 5. There is a general correlation between in vitro enzyme selectivity and selectivity in cells. For example, Compound 2 or MS-275 are both class I HDAC inhibitors in vitro and also potent inhibitors in cells overexpressing HDAC1 but not cells overexpressing HDAC-6. However, SAHA, a pan inhibitor of class I/II HDACs, can inhibit both HDAC-1 and HDAC-6 in cells. We also demonstrate that Compound 5, which shows HDAC-6 selectivity in vitro, also exhibits HDAC-6 selectivity in whole cells. Thus, we could use a pan-substrate against class I/II enzyme to analyze potency and isotype-specific inhibitory activity of a pan- or isotype-selective inhibitor in a cell population where one or a few HDAC isotypes are abundant.

TABLE 5
Whole cell deacetylase IC50 (uM) in human 293T cells overexpressing
HDAC1 or HDAC6 as well as their IC50 against recombinant HDAC-1 or
HDAC-6 in vitro

| | |
|---|---|
| IC50 in cells (uM) | IC50 (uM) in vitro |

| | 293T-HD1 | 293T-HD6 | HD1 | HD6 |
|---|---|---|---|---|
| SAHA | 4 | 5 | 0.1 | 0.1 |
| MS-275 | 4 | >100 | 0.4 | >20 |
| Compound 2 | 0.5 | >100 | 0.1 | >20 |
| Compound 5 | 21 | 3 | 0.5 | 0.02 |

results are mean from at least 3 independent assays

### EXAMPLE 25

### Assessment of Deacetylase Activity Using Bodily Fluids

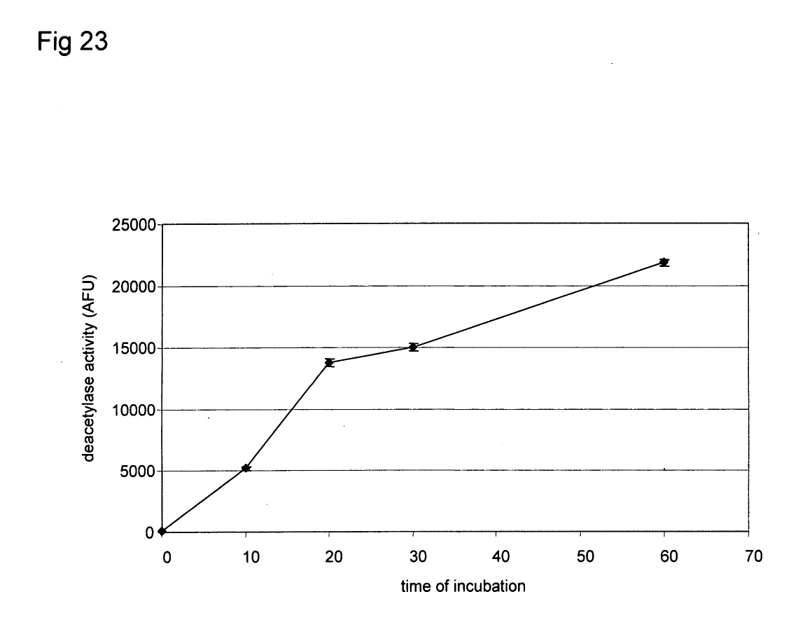

CD-1 Mouse blood was collected in heparin tubes and cells were counted by Coulter counter (Beckman Coulter, Ville St. Laurent, Quebec). The amount of whole blood containing 1.6×10E6 white blood cells was aliquoted and the volume was brought up to 200 ul with RPMI (+10% FBS). Boc-Ac-Lys-AMC was added to a final concentration of 300 uM. After various amounts of time, the mix was spun (400×g for 5 min), and 50 ul of the supernatant (serum) was transferred to a 96-well plate. The amount of deacetylated product Boc-Lys-AMC present in the supernatant was detected by adding an equal volume of the developer mix and reading after 15 minutes incubation (as described in Example 3). The results are shown in FIG. 23. This finding is consistent with our observation in Example 1, where not only the substrate Boc-Lys(Ac)-AMC is permeable to go inside cells, but also the deacetylated product Boc-Lys-AMC is permeable to come out from cells. Thus total HDAC activity in primary cells could be easily monitored in bodily fluid where animals were contacted with HDAC substrates ex vivo.

### EXAMPLE 16

### Assessment of Protein Deacetylase Activity In Vivo Using Bodily Fluids

CD-1 mice (6 per group) or rats (6 per group) are treated with a cell permeable pan-substrate at 1 to 100 mg/kg by a single i.v. administration. Three of the mice (or rats) are then treated with a pan-inhibitor of a protein deacetylase family. At times thereafter, blood is taken, plasma separated and analyzed for the quantity of the detectable reporter molecule. The quantity of reporter molecule in the plasma from inhibitor-treated mice is compared with the quantity in the plasma of the untreated mice.

### Equivalents

Those skilled in the art will recognize, or be able to ascertain, using no more than routine experimentation, many equivalents to the specific embodiments of the invention described herein. Such equivalents are intended to be encompasssed by the following claims.

What is claimed is:

1. A method for assessing total protein deacetylase activity of a protein deacetylase family in whole cells or one or more members thereof ex vivo comprising providing whole cells from a mammal; contacting the whole cells with a cell-permeable pan-substrate or isotype-specific substrate for the protein deacetylase family or the one or more members thereof, wherein deacetylation of the substrate by the protein deacetylase family or the one or more members thereof generates a detectable reporter molecule; and quantitating the detectable reporter molecule.

2. The method of claim 1, wherein the quantity of the detectable reporter molecule is measured against a control standard for the protein deacetylase family or the one or more member thereof.

3. The method of claim 1, wherein the protein deacetylase family is a histone deacetylase (HDAC) family.

4. The method of claim 1, wherein the protein deacetylase family is a Sir-2 family.

5. A method for assessing isotype-specific activity of one or more member of a protein deacetylase family from whole cells ex vivo, wherein the one or more isotype of the protein deacetylase family provides a majority of the total deacetylase activity, comprising providing whole cells from a mammal; contacting the whole cells with a cell-permeable pan-substrate for the protein deacetylase family or a cell permeable isotype-specific inhibitor of the one or more member of the protein deacetylase family, wherein deacetylation of the substrate by the protein deacetylase generates a detectable reporter molecule; contacting a first aliquot of the cells with an isotype-specific inhibitor of the one or more protein deacetylase that provides a majority of the total deacetylase activity; providing a second aliquot of the whole cells which is not contacted with the isotype-specific inhibitor of the one or more protein deacetylase that provides a majority of the total deacetylase activity; quantitating the detectable reporter molecule in the first and second aliquots; and comparing the detectable reporter molecule in the first and second aliquots.

6. The method of claim 5, wherein the quantity of the detectable reporter molecule is measured against a control standard for the protein deacetylase family or the one or more member thereof.

7. The method of claim 5, wherein the protein deacetylase family is a histone deacetylase (HDAC) family.

8. The method of claim 5, wherein the protein deacetylase family is a Sir-2 family.

9. The method of claim 5, wherein the whole cells have been transfected with a gene or genes expressing the one or more isotype prior to quantitating the detectable reporter molecule.

10. A method for assessing the isotype-specific activity of one or more member of a protein deacetylase family ex vivo, comprising providing whole cells from a mammal; contacting the whole cells with a cell-permeable isotype-specific substrate for the one or more member of a protein deacetylase family, wherein deacetylation of the substrate by the one or more protein deacetylase generates a detectable reporter molecule; and quantitating the detectable reporter molecule.

11. The method of claim 9, wherein the quantity of the detectable reporter molecule is measured against a control standard for the protein deacetylase family or the one or more members therof.

12. The method of claim 9, wherein the protein deacetylase family is a histone deacetylase (HDAC) family.

13. The method of claim 9, wherein the protein deacetylase family is a Sir-2 family.

14. A method for assessing the activity of a candidate pan-inhibitor of a protein deacetylase family or one or more members thereof in whole cells ex vivo, comprising providing whole cells from a mammal; contacting the whole cells with a cell-permeable pan-substrate for the protein deacetylase family or an isotype-specific substrate, wherein deacetylation of the substrate by the protein deacetylase family or the one or more members thereof generates a detectable reporter molecule; contacting a first aliquot of the cells with a candidate pan-inhibitor of the protein deacetylase family; providing a second aliquot of the cells which is not contacted with the candidate pan-inhibitor of the protein deacetylase family; quantitating the detectable reporter molecule in the first and second aliquots; and comparing the quantity of detectable reporter molecule in the first aliquot and the second aliquot.

15. The method of claim 13, wherein the quantity of the detectable reporter molecule is measured against a control standard for the protein deacetylase family or the one or more members thereof.

16. The method of claim 13, wherein the protein deacetylase family is a histone deacetylase (HDAC) family.

17. The method of claim 13, wherein the protein deacetylase family is a Sir-2 family.

18. A method for assessing isotype-specific activity of a candidate inhibitor of one or more member of a protein deacetylase family from whole cells ex vivo, wherein the one or more isotype of the protein deacetylase family provides a majority of the total deacetylase activity; the method comprising providing whole cells from a mammal; contacting the whole cells with a cell-permeable pan-substrate for the protein deacetylase family or a cell permeable isotype-specific inhibitor of the one or more member of the protein deacetylase family, wherein deacetylation of the substrate by the protein deacetylase generates a detectable reporter molecule; contacting a first aliquot of the whole cells with the candidate isotype-specific inhibitor of the one or more protein deacetylase that provides a majority of the total deacetylase activity; providing a second aliquot of the cells which is not contacted with with the candidate isotype-specific inhibitor of the one or more protein deacetylase that provides a majority of the total deacetylase activity; quantitating the detectable reporter molecule in the first and second aliquots; and comparing the quantity of the detectable reporter molecule for each aliquot.

19. The method of claim 17 wherein the quantity of the detectable reporter molecule is measured against a control standard for the protein deacetylase family or the one or more members thereof.

20. The method of claim 17, wherein the protein deacetylase family is a histone deacetylase (HDAC) family.

21. The method of claim 17, wherein the protein deacetylase family is a Sir-2 family.

22. A method for assessing the efficacy of a pan-inhibitor of a protein deacetylase family or one or more members thereof in vivo, comprising providing whole cells from a mammal; contacting the whole cells with a pan-substrate for the protein deacetylase family or an isotype-specific substrate, wherein deacetylation of the substrate by the protein deacetylase family or the one or more members thereof generates a detectable reporter molecule; quantitating the reporter molecule; administering to the mammal the pan-inhibitor; providing whole cells from the mammal; contacting the whole cells with the pan-substrate or isotype-specific substrate; quantitating the reporter molecule; and comparing the quantity of the reporter molecule in the whole cells from the mammal before administration of the pan-inhibitor with the quantity of the reporter molecule in the whole cells after administration of the pan-inhibitor.

23. The method of claim 21, wherein the protein deacetylase family is a histone deacetylase (HDAC) family.

24. The method of claim 21, wherein the protein deacetylase family is a Sir-2 family.

25. A method for assessing the efficacy of an isotype-specific inhibitor of a protein deacetylase family in vivo, providing whole cells from a mammal; contacting the whole cells with an isotype-specific substrate for one or more member of a protein deacetylase family, wherein deacetylation of the substrate by the one or more protein deacetylase generates a detectable reporter molecule; quantitating the reporter molecule; administering to the mammal the isotype-specific inhibitor; providing whole cells from the mammal; contacting the whole cells with the isotype-specific substrate; quantitating the reporter molecule; and comparing the quantity of the reporter molecule from the whole cells after administration of the isotype-specific inhibitor with the quantity of the reporter molecule in the whole cells from the mammal before administration of the isotype-specific inhibitor.

26. The method of claim 24, wherein the protein deacetylase family is a histone deacetylase (HDAC) family.

27. The method of claim 24, wherein the protein deacetylase family is a Sir-2 family.

28. A method for assessing the efficacy of a pan-activator of a protein deacetylase family or one or more members thereof in vivo, comprising providing whole cells from a mammal; contacting the whole cells with a pan-substrate for the protein deacetylase family or an isotype-specific substrate, wherein deacetylation of the substrate by the protein deacetylase family or the one or more members thereof generates a detectable reporter molecule; quantitating the reporter molecule; administering to the mammal the pan-activator; providing whole cells from the mammal; contacting the whole cells with the pan-substrate or isotype-specific substrate; quantitating the reporter molecule; and comparing the quantity of the reporter molecule in the whole cells from the mammal before administration of the pan-activator with the quantity of the reporter molecule in the whole cells after administration of the pan-inhibitor.

29. The method of claim 27, wherein the protein deacetylase family is a histone deacetylase (HDAC) family.

30. The method of claim 27, wherein the protein deacetylase family is a Sir-2 family.

31. A method for assessing the efficacy of an isotype-specific activator of a protein deacetylase family in vivo, providing whole cells from a mammal; contacting the whole cells with an isotype-specific substrate for one or more member of a protein deacetylase family, wherein deacetylation of the substrate by the one or more protein deacetylase generates a detectable reporter molecule; quantitating the reporter molecule; administering to the mammal the isotype-specific activator; providing whole cells from the mammal; contacting the whole cells with the isotype-specific substrate; quantitating the reporter molecule; and comparing the quantity of the reporter molecule from the whole cells after administration of the isotype-specific activator with the quantity of the reporter molecule in the whole cells from the mammal before administration of the isotype-specific activator.

32. The method of claim 30, wherein the protein deacetylase family is a histone deacetylase (HDAC) family.

33. The method of claim 30, wherein the protein deacetylase family is a Sir-2 family.

34. A method for assessing the efficacy of a pan-inhibitor of total protein deacetylase of a mammal or of one or more members thereof in vivo comprising administering to the mammal a cell-permeable pan-substrate for a protein deacetylase family, wherein deacetylation of the pan-substrate or isotype-specific substrate generates a detectable reporter molecule; obtaining bodily fluids from the mammal; determining the quantity of the detectable reporter molecule in the bodily fluids; administering to the mammal a pan-inhibitor of the protein deacetylase family; administering to the mammal the pan-substrate or the isotype-specific substrate; obtaining bodily fluids from the mammal; determining the quantity of the detectable reporter molecule in the bodily fluids; and comparing the quantity of detectable reporter molecule in bodily fluids obtained prior to administration of the pan-inhibitor with the quantity of the detectable reporter molecule in bodily fluids after administration of the pan-inhibitor.

35. The method of claim 33, wherein the protein deacetylase family is a histone deacetylase (HDAC) family.

36. The method of claim 33, wherein the protein deacetylase family is a Sir-2 family.

37. A method for assessing the efficacy of an isotype-specific inhibitor of one or more member of a protein deacetylase family in a mammal in vivo comprising administering to the mammal a cell-permeable isotype-specific substrate for one or more member of a protein deacetylase family, wherein deacetylation of the isotype-specific substrate generates a detectable reporter molecule; obtaining bodily fluids from the mammal; determining the quantity of the detectable reporter molecule in the bodily fluids; administering to the mammal an isotype-specific inhibitor of the one or more member of a protein deacetylase family; administering to the mammal the isotype-specific substrate; obtaining bodily fluids from the mammal; determining the quantity of the detectable reporter molecule in the bodily fluids; and comparing the quantity of detectable reporter molecule in bodily fluids obtained prior to administration of the isotype-specific inhibitor with the quantity of the detectable reporter molecule in bodily fluids after administration of the isotype-specific inhibitor.

38. The method according to claim 36, wherein the protein deacetylase family is the histone deacetylase (HDAC) family.

39. The method according to claim 36, wherein the protein deacetylase family is the Sir2 family.

40. A method for assessing the efficacy of a pan-activator of total activity of a protein deacetylase family in a mammal or one or more members thereof in vivo comprising administering to the mammal a cell-permeable pan-substrate for a protein deacetylase family or an isotype-specific substrate, wherein deacetylation of the pan-substrate or isotype-specific substrate generates a detectable reporter molecule; obtaining bodily fluids from the mammal; determining the quantity of the detectable reporter molecule in the bodily fluids; administering to the mammal a pan-activator of the protein deacetylase family; administering to the mammal the pan-substrate or the isotype-specific substrate; obtaining bodily fluids from the mammal; determining the quantity of the detectable reporter molecule in the bodily fluids; and comparing the quantity of detectable reporter molecule in bodily fluids obtained prior to administration of the pan-activator with the quantity of the detectable reporter molecule in bodily fluids after administration of the pan-activator.

41. The method according to claim 39, wherein the protein deacetylase family is the histone deacetylase (HDAC) family.

42. The method according to claim 39, wherein the protein deacetylase family is the Sir2 family.

43. A method for assessing the efficacy of an isotype-specific activator of one or more member of a protein deacetylase family in a mammal in vivo comprising administering to the mammal a cell-permeable isotype-specific substrate for one or more member of a protein deacetylase family, wherein deacetylation of the isotype-specific substrate generates a detectable reporter molecule; obtaining bodily fluids from the mammal; determining the quantity of the detectable reporter molecule in the bodily fluids; administering to the mammal an isotype-specific activator of the one or more member of the protein deacetylase family; administering to the mammal the isotype-specific substrate; obtaining bodily fluids from the mammal; determining the quantity of the detectable reporter molecule in the bodily fluids; and comparing the quantity of detectable reporter molecule in bodily fluids obtained prior to administration of the isotype-specific activator with the quantity of the detectable reporter molecule in bodily fluids after administration of the isotype-specific activator.

44. The method of claim 42, wherein the protein deacetylase family is a histone deacetylase (HDAC) family.

45. The method according to claim 42, wherein the protein deacetylase family is the Sir2 family.

46. A method for assessing the activity of a candidate pan-activator of a protein deacetylase family or one or more members thereof in whole cells ex vivo, comprising providing whole cells from a mammal; contacting the whole cells with a cell-permeable pan-substrate for the protein deacetylase family or an isotype-specific substrate, wherein deacetylation of the substrate by the protein deacetylase family or the one or more members thereof generates a detectable reporter molecule; contacting a first aliquot of the cells with a candidate pan-activator of the protein deacetylase family; providing a second aliquot of the cells which is not contacted with the candidate pan-actvator of the protein deacetylase family; quantitating the detectable reporter molecule in the first and second aliquots; and comparing the quantity of detectable reporter molecule in the first aliquot and the second aliquot.

47. A method for assessing the activity of a candidate isotype-specififc activator of a protein deacetylase family or one or more members thereof in whole cells ex vivo, comprising providing whole cells from a mammal; contacting the whole cells with a cell-permeable pan-substrate for the protein deacetylase family or an isotype-specific substrate, wherein deacetylation of the substrate by the protein deacetylase family or the one or more members thereof generates a detectable reporter molecule; contacting a first aliquot of the cells with a candidate isotype-specific activator of one or more member of the protein deacetylase family; providing a second aliquot of the cells which is not contacted with the candidate isotype-specific activator of the one or more member of the protein deacetylase family; quantitating the detectable reporter molecule in the first and second aliquots; and comparing the quantity of detectable reporter molecule in the first aliquot and the second aliquot.
